(19) United States
(12) Patent Application Publication
(10) Pub. No.: US 2004/0132183 A1
(43) Pub. Date: Jul. 8, 2004

(54) Methods and compositions for expanding and differentiating insulin-producing cells

(76) Inventors: David William Scharp, Mission Viejo, CA (US); Paul Presley Latta, Irvine, CA (US); Margaret Coutts, Irvine, CA (US); Catherine Anne McIntyre, Aliso Viejo, CA (US)

Correspondence Address: NOVOCELL, INC., 31 TECHNOLOGY DRIVE, SUITE 100, IRVINE, CA, 92618, US

(21) Appl. No.: 10/447,325

(22) Filed: May 28, 2003

Related U.S. Application Data
(60) Provisional application No. 60/384,000, filed on May 28, 2002.

(51) Int. Cl. C12N 5/08

(52) U.S. Cl. 435/366

(57) ABSTRACT

A method of converting differentiated non-hormone producing pancreatic cells into differentiated hormone producing cells is disclosed. The method comprises two steps: first, culturing cells under conditions which convert differentiated non-hormone producing cells into stem cells; and second, culturing stem cells under conditions which provide for differentiating stem cells into hormone-producing cells. The invention provides a new source of large quantities of hormone producing cells such as insulin-producing cells that are not currently available for therapeutic uses such as the treatment of diabetes.

### RELATED APPLICATIONS

This application claims priority to U.S. Provisional Application No. 60/384000, filed May 28, 2002 which is incorporated herein by reference in its entirety.

### BACKGROUND OF THE INVENTION

1. Field of the Invention

This invention relates to the culture media, mode, conditions, and methods for converting non-insulin producing pancreas cells into stem cells that can be proliferated and differentiated into pancreatic hormone producing cells.

2. Description of the Related Art

The ability to selectively control the in vitro expansion and conversion of non-insulin producing pancreatic cells, such as acinar cells or duct cells, into insulin producing cells, would create a new treatment regime for diabetes that avoids many of the shortcomings of current diabetes treatments.

Diabetes mellitus is a disease caused by the loss of the ability to transport glucose into the cells of the body, either because not enough insulin is produced or because the response to insulin is diminished. In a healthy person, minute elevations in blood glucose stimulate the production and secretion of insulin, the role of which is to increase glucose uptake into cells, returning the blood glucose to the optimal level. Insulin stimulates liver and skeletal muscle cells to take up glucose from the blood and convert it into the energy storage molecule glycogen. It also stimulates skeletal muscle fibers to take up amino acids from the blood and convert them into protein, and it acts on adipose (fat) cells to stimulate the synthesis of fat. In diabetes, the blood stream may be saturated with glucose, but the glucose cannot reach the intracellular places where it is needed and utilized. As a result the cells of the body are starved of needed energy, which leads to the wasted appearance of many patients with poorly controlled insulin-dependent diabetes.

Prior to the discovery of insulin and its use as a treatment for diabetes, the only outcome was starvation followed predictably by death. With insulin treatment today, death still occurs with over dosage of insulin resulting in extreme hypoglycemia and coma followed by death unless reversed by the intake of glucose Death also still occurs with major under dosage of insulin leading to ketoacidosis that, if not treated properly and urgently will also result in coma and death.

While diabetes is not commonly a fatal disease thanks to the treatments available to diabetics today, none of the standard treatments can replace the body's minute-to-minute production of insulin and precise control of glucose metabolism. As a consequence, the average blood glucose levels in diabetics remain generally too high. The chronically elevated blood glucose levels cause a number of long-term complications over time. Diabetes is the leading cause of blindness, renal failure, the premature development of heart disease or stroke, gangrene and amputation, impotence, and it decreases the sufferer's overall life expectancy by one to two decades.

Diabetes mellitus is one of the most common chronic diseases in the world. In the United States, diabetes affects approximately 16 million people—more than 12% of the adult population over 45. The number of new cases is increasing by about 150,000 per year. In addition to those with clinical diabetes, there are approximately 20 million people showing symptoms of abnormal glucose tolerance. These people are borderline diabetics, midway between those who are normal and those who are clearly diabetic. Many of them will develop diabetes in time and some estimates of the potential number of diabetics are as high as 36 million or 25-30% of the adult population over 45 years.

Diabetes and its complications have a major socioeconomic impact on modern society. Of the approximately $700 billion dollars spent on healthcare in the US today, roughly $100 billion are spent to treat diabetes and its complications. Since the incidence of diabetes is rising, the costs of diabetes care will occupy an ever-increasing fraction of total healthcare expenditures unless steps are taken promptly to meet the challenge. The medical, emotional and financial toll of diabetes is enormous, and increases as the numbers of those suffering from diabetes grows.

Diabetes mellitus can be subdivided into two distinct types: Type 1 diabetes and Type 2 diabetes. Type 1 diabetes is characterized by little or no circulating insulin and it most commonly appears in childhood or early adolescence. It is caused by the destruction of the insulin-producing beta cells of the pancreatic islets. There is a genetic predisposition for Type 1 diabetes with the destruction resulting from an autoimmune attack against the beta cells, initiated by some as yet unidentified environmental event, such as a viral infection, or the action of a noninfectious agent (a toxin or a food), which triggers the immune system to react to and destroy the patient's beta cells in the pancreas. The pathogenic sequence of events leading to Type 1 diabetes is thought to consist of several steps. First, it is believed that genetic susceptibility is an underlying requirement for the initiation of the pathogenic process. Secondly, an environmental insult mediated by a virus or noninfectious agent such as toxin or food triggers the third step, the inflammatory response in the pancreatic islets (insulitis) in genetically predisposed individuals. The fourth step is an alteration or transformation of the beta cells such that they are no longer recognized as “self” by the immune system, but rather seen as foreign cells or “nonself”. The last step is the development of a full-blown immune response directed against the “targeted” beta cells, during which cell-mediated immune mechanisms cooperate with cytotoxic antibodies in the destruction of the insulin-producing beta cells. Despite this immune attack, for a period of time, the production of new beta cells is fast enough to stay ahead of the destruction by the immune system and a sufficient number of beta cells are present to control blood glucose levels. Gradually, however, the number of beta cells declines. When the number of beta cells drops to a critical level (10% of normal), blood glucose levels can no longer be controlled and the progression to total failure of insulin production is almost inevitable. It is thought that the regeneration of beta cells continues for a few years, even after functional insulin production ceases, but that the cells are destroyed as they develop maturity.

To survive, people with Type 1 diabetes must take multiple insulin injections daily and test their blood sugar by pricking their fingers for blood multiple times per day. The multiple daily injections of insulin do not adequately mimic the body's minute-to-minute production of insulin and precise control of glucose metabolism. Blood sugar levels are usually higher than normal, causing complications that include blindness, heart attack, kidney failure, stroke, nerve damage, and amputations. Even with insulin, the average life expectancy of a diabetic is 15-20 years less than that of a healthy person.

Type 2 diabetes usually appears in middle age or later and particularly affects those who are overweight. Over the past few years, however, the incidence of Type 2 diabetes mellitus in young adults has increased dramatically. In the last several years, the age of onset of Type 2 diabetes has dropped from 40 years of age to 30 years of age with those being obese, the new younger victims of this disease. In Type 2 diabetes, the body's cells that normally require insulin lose their sensitivity and fail to respond to insulin normally. This insulin resistance may be overcome for many years by extra insulin production by the pancreatic beta cells. Eventually, however, the beta cells are gradually exhausted because they have to produce large amounts of excess insulin due to the elevated blood glucose levels. Ultimately, the overworked beta cells die and insulin secretion fails, bringing with it a concomitant rise in blood glucose to sufficient levels that it can only be controlled by exogenous insulin injections. High blood pressure and abnormal cholesterol levels usually accompany Type 2 diabetes. These conditions, together with high blood sugar, increase the risk of heart attack, stroke, and circulatory blockages in the legs leading to amputation. Drugs to treat Type 2 diabetes include some that act to reduce glucose absorption from the gut or glucose production by the liver and others that stimulate the beta cells directly to produce more insulin. However, high levels of glucose are toxic to beta cells, causing a progressive decline of function and cell death. Consequently, many patients with Type 2 diabetes eventually need exogenous insulin. A recent disturbing finding is the increase in the estimate from 20% to 40% of the Type 2 diabetics that will eventually require insulin treatment.

Another form of diabetes is called Maturity Onset Diabetes of the Young (MODY). This form of diabetes is due to a genetic error in the insulin-producing cells that restricts its ability to process the glucose that enters this cell via a special glucose receptor. Beta cells in patients with MODY cannot produce insulin correctly in response to glucose, resulting in hyperglycemia and require treatment that eventually also requires insulin injections.

The currently available medical treatments for insulin-dependent diabetes are limited to insulin administration and pancreas transplantation either with whole pancreas or pancreas segments. Insulin therapy is by far more prevalent than pancreas transplantation and entails administration of insulin either conventionally, by multiple subcutaneous injections, or by continuous subcutaneous injections. Conventional insulin therapy involves the administration of one or two injections a day of intermediate-acting insulin with or without the addition of small amounts of regular insulin. The multiple subcutaneous insulin injection technique involves administration of intermediate- or long-acting insulin in then evening and/or morning as a single dose together with regular insulin prior to each meal. Continuous subcutaneous insulin infusion involves the use of a small battery-driven pump that delivers insulin subcutaneously to the abdominal wall, usually through a 27-gauge butterfly needle. With this treatment modality, insulin is delivered at a basal rate continuously throughout the day and night, with increased rates programmed prior to meals. In each of these methods, the patient is required to frequently monitor his or her blood glucose levels and adjust the insulin dose if necessary. However, controlling blood sugar is not simple. Despite rigorous attention to maintaining a health diet, exercise regimen, and always injecting the proper amount of insulin, many other factors can adversely affect a person's blood-sugar control including: Stress, hormonal changes, periods of growth, illness or infection and fatigue. People with Type 1 diabetes must constantly be prepared for life threatening hypoglycemic (low blood sugar) and hyperglycemic (high blood sugar) reactions. Insulin-dependent diabetes is a life threatening disease which requires never-ending vigilance.

In contrast to insulin administration, whole pancreas transplantation or transplantation of segments of the pancreas is known to have cured diabetes in patients. However, due to the requirement for life-long immunosuppressive therapy, the transplantation is usually performed only when kidney transplantation is required, making pancreas-only transplantations relatively infrequent operations. Although pancreas transplants are very successful in helping people with insulin-dependent diabetes improve their blood sugar to the point they no longer need insulin injections and reduce long-term complications, there are a number of drawbacks to whole pancreas transplants. Most importantly, getting a pancreas transplant involves a major operation and requires the use of life-long immunosuppressant drugs to prevent the body's immune system from destroying the pancreas that is a foreign graft. Without these drugs, the pancreas is destroyed in a matter of days. The risks in taking these immunosuppressive drugs is the increased incidence of infections and tumors that can both be life threatening in their own right. The risks inherent in the operative procedure, the requirement for life-long immunosuppression of the patient to prevent rejection of the transplant and the morbidity and mortality rate associated with this invasive procedure, illustrate the serious disadvantages associated with whole pancreas transplantation for the treatment of diabetes. Thus, an alternative to both insulin injections and pancreas transplantation would fulfill a great public health need.

Islet transplants are much simpler (and safer) procedures than whole pancreas transplants and can achieve the same effect by replacing lost beta cells. Insulin producing beta cells are found in the islets of Langerhans scattered throughout the pancreas, an elongated gland located transversely behind the stomach. The pancreas secretes between 1.5 and 3 liters of alkaline fluid containing enzymes and pro-enzymes for digestion into the common bile duct. Histologically, the pancreas is composed of three types of functional cells: a) exocrine cells that secrete their enzymes into a lumen, b) ductal cells that carry the enzymes to the gut, and c) endocrine cells that secrete their hormones into the bloodstream. The exocrine portion is organized into numerous small glands (acini) containing columnar to pyramidal epithelial cells known as acinar cells. Acinar cells comprise approximately 80% of the pancreatic cells and are responsible for secreting digestive enzymes, such as amylases, lipases, phospholipases, trypsin, chymotrypsin, aminopeptidases, elastase and various other proteins into the pancreatic duct system. The pancreatic duct system consists of an intricate, tributary-like network of interconnecting ducts that drain each secretory acinus, draining into progressively larger ducts, and ultimately draining into the main pancreatic duct. The lining epithelium of the pancreatic duct system consists of duct cells, a cell type comprising approximately 10% of pancreatic cells. Duct cell morphology ranges from cuboidal in the fine radicles draining the secretory acini to tall, columnar, mucus-secreting in the main ductal system.

The endocrine portion of the pancreas is composed of about 1 million small endocrine glands, the islets of Langerhans, scattered throughout the exocrine pancreas. Although the islet cells comprise only approximately 2% of the pancreatic cells, the islet cells are responsible for the maintenance of blood glucose levels by secreting insulin appropriately and are the most important cells in the pancreas. There are seven types of islet cells classified according to the type of endocrine hormone secreted. The beta cells of the islet produce insulin. As discussed above, when there are insufficient numbers of beta cells, or insufficient insulin secretion, regardless of the underlying reason, diabetes results. Reconstituting the islet beta cells in a diabetic patient to a number sufficient to restore normal glucose-responsive insulin production would solve the problems associated with both insulin injection and major organ transplantation.

The islet transplantation outpatient procedure allows patients to remain fully conscious under local anesthesia while the equivalent of a 2-3 milliliters of pure islet cells is piped through a small catheter to the liver. The patients can return home or to regular activities soon after the procedure. Thus, transplanting islets instead of transplanting the entire pancreas or segments thereof offers a number of ways around the risks of the whole organ transplant. However, the shortage of islet cells available for transplantation remains an unsolved problem in islet cell transplantation. Since islets form only about 2% of the entire pancreas, isolating them from the rest of the pancreas that does not produce insulin takes approximately 6 hours. Although an automated isolation method has made it possible to isolate enough islets from one pancreas to transplant into one patient, as opposed to the 5 or 6 organs previously needed to carry out one transplant, the demand for islets still exceeds the currently available supply of organs harvested from cadavers. In the United States, due to a combination of low organ donor rates and the increasing occurrence of insulin-dependent diabetes, there are only approximately 6,000 pancreases available for transplantation or islet cell isolation, while the new cases of insulin-dependent diabetes diagnosed each year number approximately 35,000 (Hering, B. J. & Ricordi, C. (1999) Graft 2, 12-27).

One solution to the problem of severe islet cell shortage is the genetic engineering of other cells to produce insulin. Genetically engineering other cells to produce insulin has already shown some success in muscle and liver cells in that they can be modified to produce proinsulin, the precursor to insulin. However, improving secretion of the insulin in these genetically engineered cells will still require considerable investigative effort and their low insulin production renders them as yet unsuitable for transplantation. Another strategy, xenotransplantation, the transplant of an organ (or tissues or cells, in the case of diabetes) from one species to another faces a number of fundamental obstacles to becoming a viable alternative to insulin injections of human transplantation. The risks associated with xenotransplantation include transfer of prions such as those causing mad cow disease (bovine spongi form encephalopathy or BSE), and transmission of animal retroviruses such as PoERV (porcine endogenous retrovirus). Another obstacle is the problem of hyperacute rejection. The more distant the two species involved in the transplant are in evolutionary terms, the more rapid and severe the rejection process when the organs of one are transplanted into the other and the need for stronger and more risky immuno suppression. Strategies involving the genetic engineering of animal islets so as to make them less likely to succumb to immune system attach and destruction poses the risk of tampering with the silent human endogenous retroviral sequences (HERVs) thousands of which are spread throughout the human genome. Activation of these sequences by recombination and the ensuing expression of HERV proteins may lead to cancer or immune system dysregulation (Romano et al., Stem Cells 2000; 18:19-39). Finally, animal and human organs and cells differ in many ways: In their anatomy or structure, production of hormones, rates of filtration, secretion and absorption of enzymes and other chemicals, in their resistance to disease, and expected longevity.

Another strategy to solve the problem of tissue availability for islet cell transplantation is the isolation of embryonic or totipotent stem cells. Totipotent stem cells are cells that are capable of growing into any other type of cell in the body, including into an entire organism. The problem with using this type of stem cell to grow as many islets as are needed to meet the demand for transplants for diabetes lies in their procurement from abortions or in vitro fertilizations with inherent ethical and political risks. Furthermore, the techniques to differentiate totipotent stem cells into normal insulin-producing cells has not been perfected and controlled in terms of their routine differentiation into insulin-producing cells in the great quantities that will be needed. Their ability to produce insulin in response to increases in glucose concentration that trigger insulin secretion in normal beta cells, indicating that they are not behaving as normal islet beta cells (Vogel, Science, 2001 292: 615-617). Finally, the use of embryonic stem cells for therapeutic purposes in patients carries the inherent danger of tumor growth. Mouse embryonic stem cells are tumorigenic when injected into adult mice, and human embryonic stem cells also demonstrate a similar tumorigenic potential when injected into immune incompetent mice. The potential use of embryonic stem cells requires the precise separation of undifferentiated stem cells from the desired differentiated progeny, a critical and as yet unattained prerequisite for clinical application (Solter and Gearhart, Science 1999, 283: 1468-1470) in order to prevent potential tumor formation.

Thus, there exists a critical unmet medical need for large numbers of non-tumorigenic human beta cells to treat millions of diabetic patients worldwide. A strategy for the large-scale production of human insulin-producing beta cells from readily available starting material such as pancreatic acinar and duct cells that are converted into clinically relevant stem cells, would overcome the obstacles faced by the current approaches.

In examining the prior art in terms of beginning with primary pancreatic cells and converting them to insulin producing cells, the experience historically falls into three categories based on the starting cells of interest: either islet cells, duct cells, or acinar cells. There are many prior experiences starting with islet cells to grow and expand the islet cell mass in vitro. Essentially all of these approaches isolate purified islets and place them predominantly into adherent culture systems in which the islets loose their islet phenotype, plate out as single cells, and grow to confluence. Most efforts to induce direct differentiated islet cell replication in vitro have shown limited capability to proliferate islet cell mass while maintaining their differentiated state. The collected experience of these studies is that in most circumstances, after a period of culture of these adherent islet cells, they lose their islet phenotype and dedifferentiate into a more primitive cell type that is poorly characterized but expands for a time in vitro. Yet, these cells invariably enter into senescence with the loss of the cultures.

It has proved very difficult to redifferentiate these more primitive cells back to differentiated islets (Nielson 92, Brelje 93, Bonner Weir 93, Otonkoski 91, Otonkoski 94). However, in one approach (Cornelius 97), the islet cultures from NOD mice were allowed to plate and then were left without media changes for several weeks. A few cells of a poorly identified epithelial cell type was all that survived and could be grown out that demonstrated the ability to proliferate and could be differentiated into islet cells with different stages of culture conditions and reagents. The resulting U.S. Pat. Nos., 5,834,308 and 6,001,647, claim these poorly described epithelial cells as stem cells that require this method of culture to isolate, grow, and develop them into functional insulin-producing cells. While demonstrating the presence of stem cells by this method of pancreatic cell adherent culture, the technique of starvation of the cells to a minimal survival, and growth and differentiation into islet cells is problematic. This approach requires extensive growth of islet cells to reach the levels required to produce large scale implants for the treatment of diabetes. There is no evidence to date that this procedure is applicable to human cells and that such a scale up is possible while retaining the differentiated phenotype of these islet cells required for a clinical product. Therefore, we have turned to an alternative approach as described in this invention that significantly differs in that it does not start with primary islet cells to form the stem cells that can be expanded and differentiated to insulin producing cells. Instead, we start with non-insulin-producing pancreatic cells, and convert them to stem cells that expand and then differentiate into islet cells.

Others have placed the islet cells into MATRIGEL, collagen, or agarose rather than the use adherent cultures (Kerr-Conte 96). This results in the formation of cystic duct structures with regression of islet tissues and growth and differentiation of duct structures and cells of ductal phenotype. The inventors of this application have also placed isolated human islets into MATRIGEL and have confirmed the induction of duct cells that replace the differentiated islet cell mass. Different matrices can also convert islet cells to duct cells, especially in the presence of HGF (Lefrbvre 98), but again fail to produce islets. While claims of islet cells forming from these structures have been made, it is unclear as to whether their origin is from residual islet tissue present in the starting cells or new insulin-producing cells. The duct structures and islet cells may also develop from a stem cell that has not as yet been specifically identified.

The next approach that has been explored is to start with pancreatic duct cells to determine the ability to form new islet cells. It is based on the observations in both developing fetal pancreas as well as adult pancreas induced to damage by disease or manipulation where one observes the formation of new islets budding off ductal structures that have led to the idea that there is a pancreatic stem cell associated with the ductal structures that can be activated by fetal development, or damage or loss to islet mass in the adult pancreas.

Starting from isolated and purified duct structures from mouse and rat pancreas and not from human pancreas (Fung, U.S. Pat. No. 6,326,201), single cells begin to form monolayers in vitro that are predominantly a mixture of fibroblasts and stromal cells. Eventually some insulin producing cells begin to appear in these adherent cultures, but remain at a low level in the monolayers. Addition of a few growth factors minimally increased numbers of insulin cells in the monolayer. But, single cells to groups of cells, called non-adhering cells (NAC) began to appear floating above the monolayer cultures that contain islet hormone cell types. These NAC's could be increased by using growth factor pulsing prior to harvesting. They also described pdx1 positive cells, some costaining with insulin which is required as a beta cell, and others with pdx1 staining only that they describe as being progenitor cells. The NAC's were also able to show glucose stimulated insulin release. They can also add different growth factors to the monolayers and induce proliferation as well as phenotype changes. They describe the use of lectins to purify these progenitor cells as they are produced. Thus, their results support the ability of purified pancreatic duct cells from large pancreatic ducts to be dedifferentiated into progenitor cells that can differentiate into insulin producing cells by the use of their specific methods. This invention differs significantly from the Fung work in that our starting pancreatic cells are human pancreatic cells and are not isolated from purified duct structures. In fact, he claims producing duct cells only from pancreatic duct tissue that he defines as including the main pancreatic duct, the accessory pancreatic duct, the dorsal pancreatic duct, and the ventral pancreatic duct. He separately defines interlobular ducts and intercalated ducts as separate entities that are not included in his definition of pancreatic duct. Our starting pancreatic tissue excludes the tissue he defines as pancreatic duct since these larger structures and parts of structures are screened out of our preparation during the cell isolation process and are not observed in the histologic sections of the starting material. The only pancreatic duct tissue staining positive for CK19 are the intercalated ducts located within acinar cell aggregates and completely surrounded by acinar cells.

Thus, our starting pancreatic cells are a mixture of acinar cells, intercalated duct cells surrounded by acinar cells, and stromal cells, that are harvested after purifying the islets out of the starting cell mixture, leaving very few islet cells in the pancreatic starting cells. In addition, our culturing techniques differ significantly with the different modes of culture, the multiple media, as well as the growth factors that are significantly different and are described below.

Another work is that of Bonner-Weir 2000 that also starts with duct enriched pancreas tissue with the statement that their approach does not actually work with the starting pancreatic cells that we are utilizing. Their culture method also relies on MATRIGEL that is not the subject of our primary approaches to permit the new cells to migrate into and form insulin-producing cells.

The third approach for developing large quantities of insulin-producing cells starts with acinar cells. Most of the early work with acinar cells was to maintain its phenotype in culture to better understand these cells (Oliver 87, Brannon 88). Then in attempting to understand the source of pancreatic cancer cells, attention turned to duct cells and the ability of acinar cells to apparently change phenotype to some sort of duct cell, as it was described. Culturing acinar cells in collagen gels, Lisle & Losdon 1990 describe the phenomenon of acinar cells losing their specific cell markers in this culture and picking up markers similar to duct cells for 6-12 days of culture, using their own monoclonal antibodies, but subsequently reverting back to their original acinar cell markers as the culture continues.

Again, interested in pancreatic cancer, Hall & Limoine 1992, describe the culture of acinar cells on plastic dishes whereby the cells began to change over 5-10 days to begin to express one of the duct cell markers CK19, but die off by 3 weeks. Arias & Bendayan 1993 cultured rat and guinea pig acinar cells on MATRIGEL with maintenance of their acinar phenotype but loss of the cells by one week. The addition of 2% DMSO to the culture of acinar cells in MATRIGEL changed the phenotype to duct-like cells that began to form cysts and tubules within the MATRIGEL. In addition, when in the cyst structures, the cells began to express CAII, a specific enzyme used by duct cells to release bicarbonate and water. Protein inhibitors prohibited the change into a duct-like phenotype. It appears that the combination of MATRIGEL and DMSO pushed the dedifferentiated islet cells on through the more primitive stage and further differentiated them into mature duct cells with a functional marker and the ability to form three dimensional structures. The question of mechanisms was raised as to whether stem cells were involved or whether this represented transdifferentiation.

Then, Bouwens 1994 studied potential duct cell markers in the neonatal rat and described that CK7 was a marker for large pancreatic ducts while CK19 was expressed in the smaller ducts, the intercalated ducts, and the centroacinar cells of the acinii. Another marker unique to the rat, CK20, marked similar cells as CK19. He also noted that while proliferation was going on, some cells next to expanding islets also expressed the CK19 or CK20. Examining mouse pancreas cells cultured on plates, Vila 1994 demonstrated human acinar cells express CK18 at the start but changed their expression to CK7 and CK19 over time with amylase levels going down. Also mucin 1 expression rose as well as another duct cell marker, CFTR, the marker for chloride transporter of duct cells. Again, the question was raised as to whether the mechanism of this change represented transdifferentiation or the involvement of stem cells. They also found that both HGF and TGFa exposure caused these cells to proliferate making the suggestion that a stem cell may be the cause and may have bearing in the development of ductal malignancies of the pancreas. But, no insulin production was observed.

Kerr-Conte 1996 demonstrated that placing purified human islets into MATRIGEL produced cystic duct-like structures that contained islet cells as small buds. It is not clear from this work as to what the source of these duct-like cells may be that could clearly proliferate, but there was no evidence of proliferation of the islet cells. Again, as previously discussed above, the suggestion that these may be dedifferentiating islet cells into duct-like cells was made, but the ability of these cells to proliferate while the differentiated cells did not proliferate raises the possibility that these cells represent stem cells. But, no insulin production was observed.

Bouwens 1998 compared the possibilities of transdifferentiation versus the role of stem cells as causing the proliferation of dedifferentiated cells from either the duct, acinar, or islet differentiated cells. While he favored the transdifferentiation mechanism due to cell markers showing the expression of the different cell types, his primary reason was because definitive stem cell markers for these cells had not yet been developed so it was not possible to specifically identify them. Yet, he acknowledged that indirect evidence can readily suggest the presence of stem cells and that the specific markers have simply not as yet been perfected. Yet again, no insulin production had been observed in his review.

Kerr-Conte 2000 and in U.S. patent application (No. 20020155598) suggests the presence of “pluripotent pancreatic stem cells” as the primary explanation of the ability to change terminally differentiated human pancreas cells to a more primitive cell type that has the ability to expand and then be differentiated into another type of specific cell that is terminally differentiated. As an accepted marker for this stem cell, she suggests the duct-like cells co-expressing CK19 and pdx1, similarly suggested by Fung, are those stem cells. She cultured a mixture of human acinar and duct cells in adherent culture showing the loss of amylase, the increase of CK19, and the increase of pdx1 expressions in the resulting duct-like cells that flattened out as monolayers. But, she was not able to show the conversion to insulin-producing cells but was able to show the new expression of a neuroendocrine cell marker, chromogranin A. In fact, her claim of pdx1 and CK19 stained cells as being evidence of precursor cells of insulin producing cells agrees with Fung and ourselves as well as with their being stem cells. But her claim that these indeed are insulin producing cells in her patent application remains unproven by her own data represented in FIGS. 4 & 5 that fails to provide any direct evidence of increased insulin production by these converted cells. Thus, she has demonstrated the presence of stem cells but fails to demonstrate their differentiation into insulin-producing cells. This is a significant difference compared to this invention where we clearly demonstrate the production of insulin-producing cells. The methods described in these two publications utilize single pancreas cells decreased in islet content, cultured in monolayers to change the acinar phenotype to the duct-like phenotype that are called ductal precursors. By her definition, these ductal precursor cells have the ability to be differentiated into insulin-producing cells. She attempts the redifferentiation by placing the ductal precursor cells into a matrix of MATRIGEL or collagen. She clearly demonstrates the ability of the ductal precursor cells to proliferate, but in the patent application, does not demonstrate the formation of any new insulin-producing cells.

There are significant differences between her techniques and those in this invention. The first step of converting the phenotype of non-insulin producing pancreatic cells to stem cells in this invention can utilize several different media in several different culture modes in addition to adherent culture using several different types of growth factors. A stem cell is formed as demonstrated by its ability to undergo replication as the intermediary, more primitive cell that carries the only makers accepted to date to identify this stem cell that are duct cell markers like CK19 and pdx1 expression in replicating cells. Her second step does not produce insulin-producing cells. In our second step, these stem cells are then differentiated into insulin producing cells by a different set of growth factors and conditions, again demonstrated in different cell culture modes. Our invention also utilizes more complex growth and differentiation factors (Table 1) than described in her publication and patent application. The normal histology and function of our new insulin-producing cells are also shown below. The definition of the stem cell used in this invention is based on the National Library of Medicine's definition that it is a cell that is not terminally differentiated that undergoes replication as well as can differentiate into more than one type of differentiated cells. Our examples show the starting non-insulin producing pancreatic cells are converted under the first set of culture conditions into stem cells that replicate and carry the CK19 and pdx1 markers. These stem cells can then be differentiated into hormone producing islet cells such as insulin or glucagon as well as into duct structures under separate differentiating conditions as described below.

Definitions:

General source of many of these definitions is OMIM, National Center for Biotechnology Information, National Library of Medicine, National Institutes of Health.

Acinar cells—pancreatic cells that make up 80% of the pancreas and produce many different enzymes including amylase, lipase, trypsin, chymotrypsin, elastase, and many others. Acinar cells can be identified by their enzyme content, by specific cytokeratins such as CK18, and by lectins against surface sialoglycoproteins. Acinar cells form spherical structural units in the pancreas called acini consisting of polarized cells that release their enzyme products into the small, centralized intercalated ducts located at the center of each acinus. Many acinar cells contain two nuclei at any time of examination of primary cells.

Duct cells—pancreatic cells making up 10% of the pancreas that define the larger interlobular and intralobular ducts as well as the smallest, intercalated ducts, that drain the pancreatic enzymes from the acini. Duct cells also produce bicarbonate and water to dilute the enzymes and alter the intestinal pH upon release into the gut from these ductal structures. Duct cells can be identified by cytokeratin subtypes such as CK19 and by the enzymes responsible for bicarbonate production.

Islet cells—endocrine cells making up 2% of the pancreas and existing as separate cell aggregates called islets that contain different cell types making different hormones. Beta cells that are 50-60% of the islet aggregate make insulin that permits glucose entry into most cells of the body. Alpha cells that are 30% of the islet make glucagon that is released during fasting to permit glucose delivery from the liver to maintain normal blood sugar. Delta cells, 10% of the islet cells, make somatostatin that fine tunes glucose levels. Pancreatic polypeptide producing cells (5-10% of the islet cells) release their hormone that alters exocrine and gastrointestinal function. In addition to these major islet cell types, there are also other islet cell types that make a variety of other hormones including GIP, VIP, gastrin, bombesin, and others. In addition, the islets contain fenestrated endothelium as a rich capillary bed into which each islet cell to releases its hormone product.

Pancreatic cells—primary pancreatic cells from human donors (or other mammalian species) that contain acinar, duct, and islet cells types as well as supportive and vascular cells.

Islet-depleted pancreatic cells—the cells remaining after the isolation of islets from a suspension of digested pancreatic cells using a discontinuous or continuous density gradient. This population is comprised mainly of acinar cells (>90%) with a small percentage of intercalated ducts within the acinar aggregates, vascular, and neuronal tissue, as well as a residual amount of contaminating islet material.

Pancreatic Acinus—any of the small spherical acinar cell structures that empty their enzyme products into the central acinar area that empties into the intercalated pancreatic ducts.

Intercalated Duct—a duct from a tubule or acinus of the pancreas that drains into an intralobular duct.

Intralobular Duct—a duct that collects pancreatic juice from the intercalated ducts and drains into an interlobular duct.

Interlobular Duct—a duct that collects pancreatic juice from intralobular ducts and drains into pancreatic ducts

Pancreatic Duct—largest of the ducts that includes the main pancreatic duct, the accessory pancreatic duct, the dorsal pancreatic duct, and the ventral pancreatic duct

Stem Cell—a cell that is not terminally differentiated that can undergo replication and can differentiate into more than one type of differentiated cell.

Cell Growth—is the replication of the cellular DNA followed by cytokinesis that can be demonstrated by BrdU or tritiated thymidine incorporation or Ki67.

Cell Expansion—used to define numbers of cells that have gone through cell division and are increasing their numbers and overall mass, rather than simply enlarging by hypertrophy.

Proliferation—rapid and repeated production of new parts or of offspring (as in a mass of cells by a rapid succession of cell divisions).

Cell Hypertrophy—used to define enlarging cells that have increased their cell volume, rather than growing by cell division.

Cell Cycle—cell growth cycle. Cells that are in cell cycle have left the resting state (Go phase) and are replicating their contents and dividing in two.

Differentiation—is used to declare that a cell has passed from a progenitor level or more basic or generalized function to one of more specific function.

Transdifferentiation—is uses to declare that a cell has changed from a level of defined function to another.

Dedifferentiation—is used to declare that a cell has passed from a level of defined function to one of less defined function or to a basic cell.

Totipotent—capable of developing into a complete organism or differentiating into any of its cells or tissues.

Pluripotent—1: not fixed as to developmental potentialities: having developmental plasticity such as a pluripotent cell or pluripotent embryonic tissue. 2: capable of affecting more than one organ or tissue.

Growth Factors (GF)—include a number of compounds that may induce cell replication. There are general GF's such as Epidermal GF (EGF) and Vascular Endothelial GF (VEGF). There are also GF's that are more specific in their action. (e.g. the action of Insulin-like GF 1 (IGF1) on islets, or erythropoietin on red blood cell progenitors).

Differentiation Factors (DF)—include a number of compounds that may induce cell type specific differentiation. There are specific differentiation factors for islet cells, for acinar cells, and for duct cells. An example for acinar cells is dexamethasone.

Dedifferentiation Factors (DDF)—include a number of factors for islet cells, for acinar cells, and for duct cells that permit the cell to lose differentiated function and change to a level of function that is lower in the lineage.

Matrix or Matrices—used to define hydrogels or polymerizable materials that hold cells in place for culture under different conditions. These include MATRIGEL, collagen, alginate, and others.

Tissue Culture Flask, Dish or Plate Substrates—used to define specific types of plastic or glass surfaces that are configured either in tissue culture flasks, petri dishes or culture plates that are used to grow cells. These surfaces are prepared such that they either promote or discourage adherent or non-adherent cell growth.

Coated Culture Flask, Dish, or Plate Surfaces—a cell culture dish coated with a thin layer of a compound.

Suspension culture—cells suspended in tissue culture medium in the absence of any support from a thin layer of a compound or any matrix.

Alpha-tocopherol—Vitamin E and Vitamin E related vitamins are chemically tocopherols. They are essential in the nutrition of various vertebrates in which their absence is associated with infertility, degenerative changes in muscle, or vascular abnormalities, are found especially in wheat germ, vegetable oils, egg yolk, and green leafy vegetables or are made synthetically, and are used chiefly in animal feeds and as antioxidants.

Apotransferrin—protein produced by oligodendricytes that is necessary for cell survival and involved in cell differentiation.

Biotin—a colorless crystalline growth vitamin C₁₀H₁₆N₂O₃S of the vitamin B complex found especially in yeast, liver, and egg yolk.

BSA—(bovine) serum albumin is a monomeric protein that comprises about one-half of the blood's serum proteins. In vivo, it plays a role in stabilizing extracellular fluid volume and functions as a carrier for steroids, fatty acids, and some hormones.

CAII—carbonic anhydrase type II, the enzyme used by duct cells to produce bicarbonate that is secreted into the pancreatic ducts to neutralize the acid in the duodenum generated by the stomach.

Calcium pantothenate—a white powdery salt C₁₈H₃₂CaN₂O₁₀ made synthetically and used as a source of pantothenic acid.

Carnitine—a quaternary ammonium compound C₇H₁₅NO₃ present especially in vertebrate muscle and involved in the transfer of fatty acids across mitochondrial membranes.

Catalase—enzyme that consists of a protein complex with hematin groups and catalyzes the decomposition of hydrogen peroxide into water and oxygen

CCK—cholecystokinin is a brain and gut peptide. In the gut, it induces the release of pancreatic enzymes and the contraction of the gallbladder. It has the capacity to stimulate insulin secretion. CCK peptides exist in multiple molecular forms (e.g., sulfated CCK8, unsulfated CCK8, and CCK4), each resulting from distinct posttranslational processing of the CCK gene product.

CFTR—cystic fibrosis transmembrane conductance regulator (CFTR) functions as a chloride channel. Mutations in the CFTR gene have been found to cause cystic fibrosis. Mutations in CFTR effect the exocrine function of the pancreas, intestinal glands, biliary tree, bronchial glands and sweat glands.

CK19—cytokeratin19 is the smallest known (40-kD) acidic keratin, one of a family of water-insoluble intermediate filaments. Different cytokeratins can be used as markers to identify certain types of epithelia and epithelial tumors. CK19 keratin is found in many types of epithelial cells, including numerous ductal and glandular epithelia. In the pancreas, it is present in ductal epithelia and absent in endocrine and exocrine cells.

CK19+ cells—cytokeratin 19 is expressed in epithelial cells in culture, in particular, in “intermediary” or transdifferentiating cells from pancreatic tissues.

Corticosteroid—any of various adrenal-cortex steroids (as corticosterone, cortisone, and aldosterone) that are divided on the basis of their major biological activity into glucocorticoids and mineralocorticoids.

Corticosterone—a colorless crystalline corticosteroid C₂₁H₃₀O₄ of the adrenal cortex that is important in protein and carbohydrate metabolism.

C-peptide—the c-peptide (“connecting” peptide) is a short polypeptide released after the conversion of proinsulin to mature insulin. Its molecular weight is 3,582 Da.

Cyclodextran—2-hyrdroypropyl-beta-cyclodextrin. A tissue culture medium additive that facilitates solublization of hydrophobic substances.

DL-alpha-tocopherol acetate—a tocopherol C₂₉H₅₀O₂ with high vitamin E potency.

DMSO—dimethyl sulfoxide (CH3)2SO—that is an agent known to induce cell differentiation, also a solvent, also a cryoprotectant for freezing living cells, also an anti-inflammatory agent for the treatment of interstitial cystitis

EGF—epidermal growth factor is a potent mitogenic factor for a variety of cultured cells of both ectodermal and mesodermal origin and has a profound effect on the differentiation of specific cells in vivo. Mature EGF is a single-chain polypeptide consisting of 53 amino acids and having a molecular mass of about 6,000.

Ethanolamine—a colorless liquid amino alcohol C₂H₇NO used especially as a solvent for fats and oils, —called also monoethanolamine.

Exendin 4—a long acting analog of GLP-1

FACS—fluorescence activated cell sorting

FCS—fetal calf serum. Blood serum recovered from an unborn cow.

FGF—The FGF superfamily consists of 23 known members, all of which contain a conserved 120 amino acid region. The FGFs were originally recognized to have proliferative activities; they are now considered to play substantial roles in development, angiogenesis, hematopoiesis, and tumorigenesis. Almost all of the FGFs isoforms have the ability to activate other isoform's receptors. This accounts for similar effects generated by different FGF subtypes.

FGF2—fibroblast growth factor 2 (FGF-basic) is a wide-spectrum mitogenic, angiogenic, and neurotrophic factor that is expressed at low levels in many tissues and cell types. FGF2 has been implicated in a multitude of physiologic and pathologic processes, including limb development, angiogenesis, wound healing, and tumor growth.

Galactose—an optically active sugar C₆H₁₂O₆ that is less soluble and less sweet than glucose and is known in dextrorotatory, levorotatory, and racemic forms.

GLP-1—Glucagon like-peptide 1 is a 30 amino acid peptide derived from the preproglucagon molecule. GLP1 enhances glucose secretion and synthesis. It renders pancreatic beta-cells ‘glucose-competent’ and may be useful in the treatment of noninsulin-dependent diabetes mellitus.

GLP-2—GLP-2 is a 33-amino acid proglucagon-derived peptide. GLP-2 maintains the integrity of the intestinal mucosal epithelium via effects on gastric motility and nutrient absorption, crypt cell proliferation and apoptosis, and intestinal permeability.

Glucose—the breakdown of carbohydrates, particularly glucose, is a major source of energy for all plant and animal cells. In diabetes, there is a diminished ability to transport glucose into the cells of the body. Blood glucose levels are abnormally high (hyperglycemia). Elevated blood glucose can lead to ketoacidosis, resulting in coma and death. Milder hyperglycemia causes long-term complications affecting the eyes, kidneys, nerves, and blood vessels.

Glutathione—a peptide C₁₀H₁₇N₃O₆S that contains one amino acid residue each of glutamic acid, cysteine, and glycine, that occurs widely in plant and animal tissues, and that plays an important role in biological oxidation-reduction processes and as a coenzyme.

Growth hormone—growth hormone (GH) is synthesized by the anterior pituitary gland. Human growth hormone has a molecular mass of 22,005 and contains 191 amino acid residues with 2 disulfide bridges. The principal biological role of growth hormone is the control of postnatal growth. It's affect is mediated largely by insulin-like growth factors.

Hb9—Homeo box-9 is one of a family of proteins that bind DNA in a sequence-specific manner and are implicated in the control of gene expression in both developing and adult tissues.

HGF—hepatocyte growth factor (also scatter factor or hepatopoietin A) has a spectrum of targets including endothelial cells and melanocytes in addition to epithelial cells such as hepatocytes. It affects diverse tissues, mediating placental growth developmental determining liver and muscle development in the embryo and B-cell proliferation and growth.

HNF3a—hepatocyte nuclear factor 3, alpha. A member of the forkhead class of transcription factors. Both HNF3A and HNF3B are expressed in tissues of endodermal origin, i.e., stomach, intestines, liver, and lung. All members of the HNF3 family as well as HNF4G and HNF6 are expressed in pancreatic beta cells

HNF6—During mouse development, Hnf6 is expressed in the epithelial cells that are precursors of the exocrine and endocrine pancreatic cells. In hnf6-null embryos, the exocrine pancreas appeared to be normal but endocrine cell differentiation was impaired. The expression of neurogenin-3, a transcription factor that is essential for determination of endocrine cell precursors, was almost abolished. Later in life, the number of endocrine cells increased but the architecture of the islets was perturbed, and the beta cells were deficient in glucose transporter-2 expression. Adult hnf6-null mice were diabetic. This suggests that Hnf6 controls embryonic pancreatic endocrine differentiation at the precursor stage and positively regulates the proendocrine gene ngn3.

HuSA—human serum albumin—see BSA (bovine serum albumin).

IBMX—3-isobutyl-1-methylxanthine A compound that inhibits cyclic AMP phosphodiesterase, which causes beta cells to release insulin.

IGF1—Insulin-like growth factor-I. Both IGF1 and IGF2 have a striking structural homology to proinsulin.

IGF2—Insulin-like growth factor 2. Both IGF1 and IGF2 have a striking structural homology to proinsulin.

Johe's N2—a serum free medium formulated for the support of multi-potential CNS stem cells is supplemented with various growth and differentiation factors

KGF—keratinocyte growth factor or FGF-7: a 28 kDa, single chain, secreted glycoprotein that has a target specificity restricted to epithelium. Adult cells known to express FGF-7 include fibroblasts, T cells, smooth muscle cells, and ovarian theca cells. In the embryo, KGF is found at many stages of development throughout the mesenchyme.

Ki67—a cell proliferation marker. This protein of unknown function is expressed during G1 of the cell cycle; it has a half-life of 60-90 minutes.

Linoleic acid—a liquid unsaturated fatty acid C₁₈H₃₂O₂ found especially in semidrying oils (as peanut oil) and essential for the nutrition of some animals—called also linolic acid

Linolenic acid—a liquid unsaturated fatty acid C₁₈H₃₀O₂ found especially in drying oils (as linseed oil) and essential for the nutrition of some animals.

Muc 1—mucin type 1, the main type of mucoprotein normally secreted by special pancreatic duct cells.

Myoinositol—a biologically active inositol that is not optically active, that is a component of the vitamin B complex and a lipotropic agent, and that occurs widely in plants, microorganisms, and higher animals including humans—called also mesoinositol

N2—Johe's N2 medium

Neuro—neurobasal medium, a neural cell culture medium

NGF—Nerve growth factor is a 12.5 kDa, nonglycosylated polypeptide 120 aa residues long. It is synthesized as a prepropeptide; its processed form is a 120 aa segment. The typical form for NGF is a 25 kDa, non-disulfide linked homodimer. Nerve growth factor is known to regulate growth and differentiation of sympathetic and certain sensory neurons

Nicotinamide—niacinamide (nicotinic acid amide) a bitter crystalline basic amide C₆H₆N₂O that is a member of the vitamin B complex and is formed from and converted to niacin in the living organism, that occurs naturally usually as a constituent of coenzymes, and that is used similarly to niacin.

PCNA+ cells—cells that label with an anti proliferating cell nuclear antigen. Proliferating cell nuclear antigen was originally correlated with the proliferative state of the cell. More recent evidence shows that PCNA may also be correlated with DNA repair.

PDGF—platelet derived growth factor. A factor released from platelets upon clotting was shown to be capable of promoting the growth of various types of cells. This factor was subsequently purified from platelets and given the name platelet-derived growth factor (PDGF). PDGF is now known to be produced by a number of cell types besides platelets and it has been found to be a mitogen for almost all mesenchymally-derived cells, i.e., blood, muscle, bone/cartilage, and connective tissue cells.

pdx-1—Pancreatic duodenal homeobox factor-1, PDX-1, is required for pancreas development, islet cell differentiation, and the maintenance of beta cell function. Also called insulin promoter factor-1 (IPF1) or IDX1 or somatostatin transcription factor-1 (STF1). PDX-1 appears to serve as a master control switch for expression of both the exocrine and endocrine pancreatic developmental programs, as revealed by gene disruption studies in which targeted deletion of the gene leads to a ‘null pancreas phenotype. PXDX-1 is initially expressed in both exocrine and endocrine cells; as pancreatic morphogenesis proceeds, it restricted to some duct cells and beta and delta cells of the islets. PDX-1 also plays a role in adult cells, regulating the insulin and somatostatin genes. Mutations in the PDX1 gene can cause pancreatic agenesis, maturity-onset diabetes of the young, and possibly type II diabetes

Placental lactogen—This peptide hormone is structurally, immunologically, and functionally similar to pituitary growth hormone It is synthesized by the placental syncytiotrophoblast

Progesterone—a female steroid sex hormone C₂₁H₃₀O₂ that is secreted by the corpus luteum to prepare the endometrium for implantation and later by the placenta during pregnancy to prevent rejection of the developing embryo or fetus and that is used in synthetic forms as a birth control pill, to treat menstrual disorders, and to alleviate some cases of infertility.

Proinsulin—the precursor of insulin. Insulin is derived from a folded, one-chain precursor that is linked by 2 disulfide bonds. Proinsulin is converted to insulin by the enzymatic removal of a segment that connects the amino end of the A chain to the carboxyl end of the B chain.

Prolactin—A growth factor with strong structural similarity to growth hormone.

PTF1—see PDX-1

PTHRP—parathyroid related protein

Putrescine—a crystalline slightly poisonous ptomaine C₄H₁₂N₂ that is formed by decarboxylation of ornithine, occurs widely but in small amounts in living things, and is found especially in putrid flesh.

Reg1—regenerating islet-derived protein also known as pancreatic stone protein

Retinyl acetate—a derivative of vitamin A

Superoxide dismutase—a metal-containing antioxidant enzyme that reduces potentially harmful free radicals of oxygen formed during normal metabolic cell processes to oxygen and hydrogen peroxide.

TGF alpha and beta—Transforming growth factors (TGFs) are biologically active polypeptides that reversibly confer the transformed phenotype on cultured cells. Alpha-TGF shows about 40% sequence homology with epidermal growth factor. TGF beta is a multifunctional peptide that controls proliferation, differentiation, and other functions in many cell types. TGFB acts synergistically with TGFA in inducing transformation. It also acts as a negative autocrine growth factor. Dysregulation of TGFB activation and signaling may result in apoptosis. Many cells synthesize TGFB and almost all of them have specific receptors for this peptide. TGFB1, TGFB2, and TGFB3 all function through the same receptor signaling systems.

Transcription Factors (TF)—Transcription factors bind to specific regulatory sequences in DNA and modulate the activity of RNA polymerase. This is the key step that regulates the process whereby genes coded in DNA are copied or transcribed into messenger RNA. Normally, the interactions of many different transcription factors determine the specific phenotype of different cell types. TF's can be positive or negative regulators of gene expression. PDX1, neurogenin 3 (ngn3), Pax4, Pax6, and others are examples of those TF's that are involved in pancreatic development and differentiation.

Triiodothyronine—a crystalline iodine-containing hormone C₁₅H₁₂I₃NO₄ that is an amino acid derived from thyroxine and is used especially in the form of its soluble sodium salt in the treatment of hypothyroidism and metabolic insufficiency—called also liothyronine, T₃.

VEGF—vascular endothelial growth factor—VEGF is a heparin-binding glycoprotein that is secreted as a homodimer of 45 kDa. One of the most important growth and survival factors for endothelium. It is structurally related to platelet-derived growth factor. VEGF induces angiogenesis and endothelial cell proliferation and it plays an important role in regulating vasculogenesis. Most types of cells, but usually not endothelial cells themselves, secrete VEGF.

Zinc sulphate—Zinc is an important trace mineral and is required for the enzyme activities necessary for cell division, cell growth, and wound healing. Zinc is also involved in the metabolism of carbohydrates. Beta cells of the pancreas have a high zinc content.

### SUMMARY OF THE INVENTION

In one embodiment, the invention is drawn to a method of converting differentiated non-hormone producing pancreatic cells into differentiated hormone-producing cells, which includes the steps of (a) culturing the differentiated non-hormone producing pancreatic cells in a first cell culture system with a first cell culture medium, under conditions which provide for converting said differentiated non-hormone producing pancreatic cells into stem cells; and b) culturing the stem cells in a second cell culture system with a second cell culture medium under conditions which provide for differentiating the stem cells into hormone-producing cells. In some embodiments, the stem cells proliferate in the first step. In some embodiments, the stem cells proliferate in the second step. In a preferred embodiment, the hormone-producing cells produce insulin. In an alternate preferred embodiment, the hormone-producing cells produce glucagon. In a preferred embodiment, the differentiated non-hormone producing pancreatic cells are acinar cells. In a preferred embodiment, the differentiated non-hormone producing pancreatic cells are seeded at a density of 10⁵ to 10⁷ cells/cm².

In a preferred embodiment, the differentiated non-hormone producing pancreatic cells in the first step are cultured with a culture mode selected from the group including: adherent, suspension and matrix; and the stem cells in the second step are cultured with a culture mode selected from the group including: adherent, suspension, and matrix.

In a preferred embodiment, the culture mode is an adherent culture mode that includes culturing cells directly on a surface of a tissue culture container or on a surface of a tissue culture container which is coated with at least one compound selected from the group including collagen, fibronectin, laminin, and hyaluronic acid.

In an alternate preferred embodiment, the culture mode is a suspension culture mode that includes culturing the differentiated non-hormone producing pancreatic cells in suspension in the culture medium.

In another alternate preferred embodiment, the culture media is a matrix culture mode that includes culturing the differentiated non-hormone producing pancreatic cells interspersed in a crosslinked polymerizable gel. In a more preferred embodiment, the differentiated non-hormone producing pancreatic cells are seeded at a density of 10⁴ to 10⁸ cells/ml in a hydrogel. In a most preferred embodiment, the hydrogel is alginate.

In one embodiment, the culture medium in the first step includes serum and a basal medium selected from the group including Dulbecco's Modified Eagle's medium (DMEM), Medium 199 (M199), Ham's F12 Nutrient Mixture (Ham's F12), RPMI 1640 Medium, CMRL medium, and mixtures thereof. In a more preferred embodiment, the culture medium in the first step further includes at least three compounds selected from the group including insulin, transferrin, selenium, zinc sulphate, glutathione, ethanolamine, cyclodextrin, biotin, alpha Tocopherol, calcium pantothenate, myoinositol, nicotinamide, IGF1, Prolactin, exendin, EGF, VEGF, KGF, and HGF.

In one embodiment, the culture medium in the first step includes a basal medium without serum selected from the group including Dulbecco's Modified Eagle's medium (DMEM), Medium 199 (M199), Ham's F12 Nutrient Mixture (Ham's F12), RPMI 1640 Medium, CMRL medium, and mixtures thereof. In a more preferred embodiment, the culture medium in the first step further includes at least three compounds selected from the group including insulin, transferrin, selenium, zinc sulphate, glutathione, ethanolamine, cyclodextrin, biotin, alpha Tocopherol, calcium pantothenate, myoinositol, nicotinamide, IGF1, Prolactin, exendin, EGF, VEGF, KGF, and HGF.

In one embodiment, the culture medium in the second step includes a basal medium without serum selected from the group including Dulbecco's Modified Eagle's medium (DMEM), Medium 199 (M199), Ham's F12 Nutrient Mixture (Ham's F12), RPMI 1640 Medium, CMRL medium, neurobasal medium, Johe's N2 medium, and mixtures thereof. In a preferred embodiment, the culture medium in the second step further includes insulin, transferrin, and selenium. In a more preferred embodiment, the culture medium in the second step further includes at least two compounds selected from the group including glutathione, ethanolamine, biotin, alpha Tocopherol (Vitamin E), and albumin (human or bovine). In a most preferred embodiment, the culture medium in the second step further includes at least two compounds selected from the group including L-carnitine, corticosterone, D(+) galactose, linoleic acid, linolenic acid, progesterone, putrescine, retinly acetate, triodo-1-thyronin (T3), DL-α-tocopherol acetate, catalase, superoxide dismutase, apotransferrin and bFGF.

In one embodiment, the culture medium in the first step includes serum and a basal medium selected from the group including Dulbecco's Modified Eagle's medium (DMEM), Medium 199 (M199), Ham's F12 Nutrient Mixture (Ham's F12), RPMI 1640 Medium, CMRL medium, and mixtures thereof. In a more preferred embodiment, the culture medium in the second step further includes at least two compounds selected from the group including glutathione, ethanolamine, biotin, alpha Tocopherol (Vitamin E), and albumin (human or bovine).

In one embodiment, the culture medium in the second step includes a basal medium without serum selected from the group including Dulbecco's Modified Eagle's medium (DMEM), Medium 199 (M199), Ham's F12 Nutrient Mixture (Ham's F12), RPMI 1640 Medium, CMRL medium, neurobasal medium, Johe's N2 medium, and mixtures thereof. In a preferred embodiment, the culture medium in the second step further includes at least two compounds selected from the group including L-carnitine, corticosterone, D(+) galactose, linoleic acid, linolenic acid, progesterone, putrescine, retinly acetate, triodo-1-tyronin (T3), DL-α-tocopherol acetate, catalase, superoxide dismutase, apotransferrin and bFGF.

In one embodiment, the culture medium in the second step includes a basal medium without serum selected from the group including Dulbecco's Modified Eagle's medium (DMEM), Medium 199 (M199), Ham's F12 Nutrient Mixture (Ham's F12), RPMI 1640 Medium, CMRL medium, neurobasal medium, Johe's N2 medium, and mixtures thereof. In a preferred embodiment, the culture medium in the second step further includes insulin, transferrin, and selenium. In a more preferred embodiment, the culture medium in the second step further includes at least two compounds selected from the group including L-carnitine, corticosterone, D(+) galactose, linoleic acid, linolenic acid, progesterone, putrescine, retinly acetate, triodo-1-thyronin (T3), DL-α-tocopherol acetate, catalase, superoxide dismutase, apotransferrin and bFGF.

In one embodiment, the invention is drawn to a method of converting differentiated non-hormone producing pancreatic cells into stem cells including culturing the differentiated non-hormone producing pancreatic cells in a cell culture system with a cell culture medium, under conditions which provide for converting the differentiated non-hormone producing pancreatic cell into stem cells. In a preferred embodiment, the stem cells a preferred embodiment, the cells are seeded at a density of 10⁵ to 10⁷ cells/cm².

Preferably, the differentiated non-hormone producing pancreatic cells include pancreatic acinar cells. More preferably, the acinar cells are in a pancreatic cell mixture.

In a preferred embodiment, the differentiated non-hormone producing pancreatic cells are cultured with a culture mode selected from the group including adherent, suspension and matrix. In a more preferred embodiment, the culture mode is an adherent culture mode that includes culturing the pancreatic cell mixture directly on a surface of a tissue culture container or on a surface of a tissue culture container which is coated with at least one compound selected from the group including collagen, fibronectin, laminin, and hyaluronic acid.

In an alternate preferred embodiment, the culture mode is a suspension culture mode that includes culturing the pancreatic cell mixture in suspension in the culture medium.

In yet another alternate preferred embodiment, the culture mode is a matrix culture mode that includes culturing the pancreatic cell mixture interspersed in a crosslinked polymerizable gel. Preferably, the pancreatic cell mixture is seeded at a density of 10⁴ to 10⁸ cells/ml in a hydrogel. More preferably, the hydrogel is alginate.

In a preferred embodiment, the culture medium includes serum and a basal medium selected from the group including Dulbecco's Modified Eagle's medium (DMEM), Medium 199 (M199), Ham's F12 Nutrient Mixture (Ham's F12), RPMI 1640 Medium, CMRL medium, and mixtures thereof. In a more preferred embodiement, the culture medium in the first step further includes at least three compounds selected from the group including insulin, transferrin, selenium, zinc sulphate, glutathione, ethanolamine, cyclodextrin, biotin, alpha Tocopherol, calcium pantothenate, myoinositol, nicotinamide, IGF1, Prolactin, exendin, EGF, VEGF, KGF, and HGF.

In a preferred embodiment, the culture medium in the first step includes a basal medium without serum selected from the group including Dulbecco's Modified Eagle's medium (DMEM), Medium 199 (M199), Ham's F12 Nutrient Mixture (Ham's F12), RPMI 1640 Medium, CMRL medium, and mixtures thereof. In a more preferred embodiment, the culture medium in the first step further includes at least three compounds selected from the group including insulin, transferrin, selenium, zinc sulphate, glutathione, ethanolamine, cyclodextrin, biotin, alpha Tocopherol, calcium pantothenate, myoinositol, nicotinamide, IGF 1, Prolactin, exendin, EGF, VEGF, KGF, and HGF.

In one embodiment, the invention is drawn to a method of culturing stem cells into differentiated hormone-producing cells, including culturing the stem cells in a cell culture system with a cell culture medium whereby the stem cells are differentiated into hormone-producing cells wherein the culture medium includes basal medium without serum and at least three compounds selected from the group including glutathione, ethanolamine, biotin, alpha Tocopherol (Vitamin E), albumin (human or bovine), L-carnitine, corticosterone, D(+) galactose, linoleic acid, linolenic acid, progesterone, putrescine, retinly acetate, triodo-1-thyronin (T3), superoxide dismutase, apotransferrin and bFGF. Preferably, the stem cells proliferate. In one preferred embodiment, the hormone-producing cells produce insulin. In an alternate preferred embodiment, the hormone-producing cells produce glucagon. Preferably, the stem cells are seeded at a density of 10⁵ to 10⁷ cells/cm².

In one embodiment, the stem cell is cultured with a culture mode selected from the group including adherent, suspension and matrix. In a preferred embodiment, the culture mode is an adherent culture mode that includes culturing stem cells directly on a surface of a tissue culture container or a surface of a tissue culture container which is coated with at least one compound selected from the group including collagen, fibronectin, laminin, and hyaluronic acid.

In an alternate preferred embodiment, the culture mode is a suspension culture mode that includes culturing the stem cells in suspension in the culture medium.

In another alternate preferred embodiment, the culture mode is a matrix culture mode that includes culturing the stem cells interspersed in a crosslinked polymerizable gel. Preferably, the stem cells are seeded at a density of 10⁴ to 10⁸ cells/ml in a hydrogel. More preferably, the hydrogel is alginate.

In a preferred embodiment, the basal medium without serum is selected from the group including Dulbecco's Modified Eagle's medium (DMEM), Medium 199 (M199), Ham's F12 Nutrient Mixture (Ham's F12), RPMI 1640 Medium, CMRL medium, neurobasal medium, Johe's N2 medium, and mixtures thereof. In a more preferred embodiment, the culture medium further includes insulin, transferrin, and selenium. In a yet more preferred embodiment, the culture medium includes at least two compounds selected from the group including glutathione, ethanolamine, biotin, alpha Tocopherol (Vitamin E), and albumin (human or bovine). In a most preferred embodiment, the culture medium further includes at least two compounds selected from the group including L-carnitine, corticosterone, D(+) galactose, linoleic acid, linolenic acid, progesterone, putrescine, retinly acetate, triodo-1-thyronin (T3), α-tocopherol, catalase, superoxide dismutase, apotransferrin and bFGF.

In a preferred embodiment, the basal medium without serum is selected from the group including Dulbecco's Modified Eagle's medium (DMEM), Medium 199 (Ml99), Ham's F12 Nutrient Mixture (Ham's F12), RPMI 1640 Medium, CMRL medium, neurobasal medium, Johe's N2 medium, and mixtures thereof. In a more preferred embodiment, the culture medium includes at least two compounds selected from the group including glutathione, ethanolamine, biotin, alpha Tocopherol (Vitamin E), and albumin (human or bovine).

In a preferred embodiment, the basal medium without serum is selected from the group including Dulbecco's Modified Eagle's medium (DMEM), Medium 199 (M199), Ham's F12 Nutrient Mixture (Ham's F12), RPMI 1640 Medium, CMRL medium, neurobasal medium, Johe's N2 medium, and mixtures thereof. In a more preferred embodiment, the culture medium further includes at least two compounds selected from the group including L-carnitine, corticosterone, D(+) galactose, linoleic acid, linolenic acid, progesterone, putrescine, retinly acetate, triodo-1-thyronin (T3), α-tocopherol, catalase, superoxide dismutase, apotransferrin and bFGF.

In a preferred embodiment, the basal medium without serum is selected from the group including Dulbecco's Modified Eagle's medium (DMEM), Medium 199 (M199), Ham's F12 Nutrient Mixture (Ham's F12), RPMI 1640 Medium, CMRL medium, neurobasal medium, Johe's N2 medium, and mixtures thereof. In a more preferred embodiment, the culture medium further includes insulin, transferrin, and selenium. In a most preferred embodiment, the culture medium further includes at least two compounds selected from the group including L-carnitine, corticosterone, D(+) galactose, linoleic acid, linolenic acid, progesterone, putrescine, retinly acetate, triodo-1-thyronin (T3), α-tocopherol, catalase, superoxide dismutase, apotransferrin and bFGF.

### BRIEF DESCRIPTION OF THE DRAWINGS

FIG. 1. Hematoxylin and eosin, and CK19 staining of cultured pancreatic cells

FIG. 2. Hematoxylin and eosin staining of isolated pancreatic cells (day 0)

FIG. 3. Anti-CK19 staining of isolated pancreatic cells (day 0)

FIG. 4. Anti-PCNA staining of isolated pancreatic cells (day 0)

FIG. 5. Anti-Insulin staining of isolated pancreatic cells (day 0)

FIG. 6. Hematoxylin and eosin staining of pancreatic cells in adherent culture (day 7)

FIG. 7. Anti-CK19 staining of pancreatic cells in adherent culture (day 7)

FIG. 8. Anti-PCNA staining of pancreatic cells in adherent culture (day 7)

FIG. 9. Anti-Insulin staining of pancreatic cells in adherent culture (day 7)

FIG. 10. Anti-Ki67 staining of isolated pancreatic cells (day 0)

FIG. 11. Anti-Ki67 staining of pancreatic cells in adherent culture in RPMI+10% FBS for 7 days

FIG. 12. Anti-Ki67 staining of pancreatic cells in adherent culture in Novocell medium for 7 days

FIG. 13. Anti-Ki67 staining of pancreatic cells in adherent culture in RPMI+10% FBS for 7 days followed by Johe's N2 medium+FGF for 7 days

FIG. 14. Anti-Ki67 staining of pancreatic cells in adherent culture in Johe's N2 medium+FGF for 7 days followed by Johe's N2 medium+Nicotinamide for 7 days

FIG. 15. Anti-Ki67 staining of pancreatic cells in adherent culture in Novocell medium for 7 days followed by Novocell medium+additional insulin for 7 days

FIG. 16. Anti-Ki67 staining of pancreatic cells in adherent culture in RPMI+10% FBS for 7 days followed by Novocell medium for 14 days

FIG. 17. Anti-Ki67 staining of pancreatic cells in adherent culture in RPMI+10% FBS for 7 days followed by Johe's N2 medium+FGF for 14 days.

FIG. 18. Anti-Ki67 staining of pancreatic cells in adherent culture in RPMI+10% FBS for 7 days followed by Johe's N2 medium+FGF for 7 days followed by Johe's N2 medium+Nicotinamide for 7 days.

FIG. 19. Induction of insulin release from pancreatic cells cultured in different medium for 14-21 days.

FIG. 20. Hematoxylin and eosin staining of isolated pancreatic cells (day 0)

FIG. 21. Anti-CK19 staining of isolated pancreatic cells (day 0)

FIG. 22. Anti-PCNA staining of isolated pancreatic cells (day 0)

FIG. 23. Anti-Insulin staining of isolated pancreatic cells (day 0)

FIG. 24. Hematoxylin and eosin staining of pancreatic cells cultured in suspension for 5 days.

FIG. 25. Anti-CK19 staining of pancreatic cells cultured in suspension for 5 days.

FIG. 26. Anti-PCNA staining of pancreatic cells cultured in suspension for 5 days.

FIG. 27. Anti-Insulin staining of pancreatic cells cultured in suspension for 5 days.

FIG. 28. Hematoxylin and eosin staining of pancreatic cells cultured in suspension for 17 days.

FIG. 29. Anti-CK19 staining of pancreatic cells cultured in suspension for 17 days.

FIG. 30. Anti-PCNA staining of pancreatic cells cultured in suspension for 17 days.

FIG. 31. Anti-Insulin staining of pancreatic cells cultured in suspension for 17 days.

FIG. 32. Hematoxylin and eosin staining of isolated pancreatic cells (day 0)

FIG. 33. Anti-CK19 staining of isolated pancreatic cells (day 0)

FIG. 34. Anti-PCNA staining of isolated pancreatic cells (day 0)

FIG. 35. Anti-pdx-1 staining of isolated pancreatic cells (day 0)

FIG. 36. Anti-Insulin staining of isolated pancreatic cells (day 0)

FIG. 37. Hematoxylin and eosin staining of pancreatic cells cultured in suspension for 7 days.

FIG. 38. Anti-CK19 staining of pancreatic cells cultured in suspension for 7 days.

FIG. 39. Anti-PCNA staining of pancreatic cells cultured in suspension for 7 days.

FIG. 40. Anti-pdx-1 staining of pancreatic cells cultured in suspension for 7 days.

FIG. 41. Anti-Insulin staining of pancreatic cells cultured in suspension for 7 days.

FIG. 42. Hematoxylin and eosin staining of pancreatic cells cultured in syspension for 7 days followed by 7 days in matrix culture

FIG. 43. Anti-CK19 staining of pancreatic cells cultured in suspension for 7 days followed by 7 days in matrix culture.

FIG. 44. Anti-PCNA staining of pancreatic cells cultured in suspension for 7 days followed by 7 days in matrix culture.

FIG. 45. Anti-pdx-1 staining of pancreatic cells cultured in suspension for 7 days followed by 7 days in matrix culture.

FIG. 46. Anti-Insulin staining of pancreatic cells cultured in suspension for 7 followed by 7 days in matrix culture.

FIG. 47. Hematoxylin and eosin staining of pancreatic cells explanted from the peritoneum of a diabetic immunocompromised mouse previously implanted with cultured pancreatic cells

FIG. 48. Hematoxylin and eosin staining of isolated islet cells (day 0)

FIG. 49. Anti-PCNA staining of isolated islet cells (day 0)

FIG. 50. Anti-Insulin staining of isolated islet cells (day 0)

FIG. 51. Anti-CK19 staining of isolated islet cells (day 0)

FIG. 52. Anti-PCNA staining of cultured islet cells (day 25)

FIG. 53 Anti-CK19 staining of cultured islet cells (day 25)

FIG. 54 Anti-Insulin staining of cultured islet cells (day 25)

### DETAILED DESCRIPTION OF THE PREFERRED EMBODIMENT

In one embodiment, the invention is drawn to a method for producing a hormone producing cell from a differentiated cell type that does not produce a hormone. Preferably, the differentiated cell type is a pancreatic cell. Preferably, the cells are islet-depleted pancreatic cells. In alternate embodiments, the cell source may be epithelial cells or stem cells. More preferably, the differentiated cell type is an acinar cell.

In one aspect the differentiated non-insulin producing pancreatic cell is converted to a stem cell. For purposes of the present disclosure, a stem cell is defined as a non-terminally differentiated cell that can replicate itself. In addition, a stem cell has the ability to produce two or more different differentiated cell types without undergoing de-differentiation. In a preferred embodiment, the stem cell is cultured under conditions to provide a hormone producing cell.

The hormone-producing cell produced in one aspect of the present invention preferably produces one or more of the hormones produced by islet cell. More preferably, the hormone-producing cell produces insulin.

In a preferred embodiment, hormone-producing cells are produced from a differentiated non-hormone producing cell. The hormone-producing cells are preferably islet cells and the hormone produced is preferably insulin. In a preferred embodiment, the non-differentiated, non-hormone-producing cell is a pancreatic cell.

Conceptually, the culturing is carried out in two steps. Without being limited by theory, Applicants believe that in a first step, the differentiated cells are dedifferentiated to stem cells. In a second step, re-differentiation of the stem cells to hormone-producing cells occurs. Overall, a cell which starts as a differentiated non-hormone producing cell is converted through a stem cell into a differentiated hormone-producing cell. This conversion takes place in the presence or absence of cell expansion.

The culture mode for the first and second steps may be any culture mode known to those skilled in the art. Preferably, the culture mode is selected from adherent, matrix or suspension culture or a combination. The cell culture mode for the second step may be the same as or different from the cell culture mode for the first step. Furthermore, the first step may use one, two, three or more than three different culture modes. Additionally, the second step may use one, two three or more than three different culture modes.

In a preferred embodiment, adherent culture is performed by allowing the cells to adhere to a culture surface. Optionally, this process may be facilitated by coating the tissue culture surface with a compound or composition to increase adherence of the cells to the surface. Such means include but are not limited to collagen, fibronectin, laminin, and hyaluronic acid. In some embodiment, non-adherent cells from an adherent culture are harvested and cultured as suspension culture cells.

In yet another preferred embodiment, the cells are cultured in a matrix culture. Matrix culture may be performed using hydrogels including but not limited to Matrigel™ (Becton Dickinson Corp.), collagen, and the like. In a most preferred embodiment, alginate is used to form a matrix. Aqueous solutions of alginate form a gel at room temperature in the presence of certain cations, especially calcium. This gel matrix can be reliquified by adding chelating agents (e.g. citrate). Cells entrapped in alginate beads are easily manipulated with less chance of physical damage from handling. In yet another preferred embodiment, suspension culture is used.

The cell culture period used for each of the first step and the second step is preferably 2-30 days, more preferably, 3-21 days and yet more preferably, 4-7 days and most preferably 5-7 days for each step. The culture period for the first step may be longer or shorter than the culture period for the second step. In one preferred embodiment, the cell culture period for the first step is about 5-10 days and the cell culture period for the second step is about 5-18 days. In some embodiments, more than one culture mode and/or culture media is used for each step.

Accordingly, a preferred aspect of the invention are methods and compositions for the large scale expansion of acinar cells and the large scale conversion of acinar cells into hormone-producing cells. Preferably, the hormone produced is insulin but other hormones are also encompassed within the invention, particularly hormones from islet cells.

Culture media used for the practice of certain embodiments of the invention are described below. In preferred embodiments, a specific tissue culture media (Novocell Basal Media [NCBM]) that has basic components and is the basal media to which different cell growth and differentiation factors are added is used. However, other culture media are also within the scope of this invention. Novocell Basal Media and other media are described below. Generally, a different culture media is used for the first step leading to the formation of stem cells than the second step which is the formation of the hormone-producing cells. In some embodiments, more than one culture media is used for each step.

The components of the basal media as well as the potential growth and differentiation factors are shown in Table 1:

TABLE 1

| | | | | Potential | |
|---|---|---|---|---|---|
| | | Potential Growth | | Differentiation | |
| Component | Range | Factors | Range | Factors | Range |
| Media 199 | 45% volume | Epidermal factor | 0.1-50 ng/ml | Nicotinamide | 0.1-5 mM |
| | | (EGF) | | | |
| Hams F12 Media | 45% volume | Vascular Endothelial | 0.1-10 ng/ml | Secretin | 0.1-50 ng/ml |
| | | Growth Factor | | | |
| | | (VEGF) | | | |
| Tissue Culture water | 10% volume | Fibroblastic Growth Factor | 0.1-50 ng/ml | Islet-like Growth | 0.1-50 ng/ml |
| | | (FGF) | | Factor II | |
| | | | | (IGFII) | |
| ZnSo₄ | 5-50 μM | Platelet Derived Growth | 0.1-50 ng/ml | Transforming Growth | 0.1-50 ng/ml |
| | | Factor (PDGF) | | Factor α | |
| | | | | (TGF-α) | |
| HEPES | 2-20 mM | Hepatocyte growth factor | 0.1-50 ng/ml | Parathyroid Hormone | 0.1-50 ng/ml |
| | | (HGF) | | Related Protein | |
| | | | | (PTHRP) | |
| Sodium Pyruvate | 0.5-5 mM | Exendin-4 | 0.05-5 ng/ml | Hepatocyte Growth | 0.1-50 ng/ml |
| | | | | Factor | |
| Insulin | 1-10 mg/L | Insulin-like Growth | 0.1-50 ng/ml | Glucagon-like Peptide-1 | 0.1-50 ng/ml |
| | | Factor I | | (GLP-1) | |
| | | (IGF-1) | | | |
| Human transferrin | 0.5-5 mg/L | Glucose | 8-20 mM | | |
| Ethanolamine | 0.1-3 mg/l | Pancreatic Regenerating | 0.1-50 ng/ml | | |
| | | Factor | | | |
| | | (Reg I) | | | |
| Human Serum | 10-30 mg/ml | Cholecystokinin | 0.1-50 ng/ml | | |
| Albumin | | (CCK) | | | |
| Sodium Selenite | 0.2-5 μg/ml | Pancreatic Polypeptide | 1-20 mg/L | | |
| Linoleic Acid | 0.2-5 μg/ml | Somatostatin | 1-20 mg/ml | | |
| Oleic Acid | 0.2-5 pg/ml | Prolactin | 0.05-25 ng/ml | | |
| Cyclodextrin | 100-750 mg/ml | Placental Lactogen | 0.05-25 ng/ml | | |
| Biotin | 0.05-0.75 μM | Transforming Growth | 0.1-50 ng/ml | | |
| | | Factor β | | | |
| | | (TGF-β) | | | |
| Glutamine | 2-15 mM | Nerve Growth Factor | 0.1-50 ng/ml | | |
| | | (NGF) | | | |
| α-tocopherol | 2-50 IU/ml | Kertinocyte Growth Factor | 0.1-50 ng/ml | | |
| | | (KGF) | | | |
| Calcium Pantothenate | 5-25 mM | | | | |
| Myoinositol | 0.05-0.75 mM | | | | |
| Sodium Hydroxide | 2 M as required for pH = | | | | |
| | 7.2-7.5 | | | | |

TABLE 2
Composition of culture media

| | | | | | Neurobasal + | Stem cell |
|---|---|---|---|---|---|---|
| | Novocell | RPMI | CMRL | Johe's N2* | supplements* | medium |
| Base | | | | | | |
| M199 | ● | | | | | |
| DMEM | | | | ● | | ● |
| RPMI | | ● | | ● | | |
| CMRL-1066 | | | ● | | | |
| F12 | ● | | | | | ● |
| Neurobasal | | | | | ● | |
| Added Supplements | | | | | | |
| FBS | | ● | ● | | | |
| Bovine serum | | | | ● | ● | |
| Albumin | | | | | | |
| Human Serum | ● | | | | | ● |
| Albumin | | | | | | |
| Alpha Tocopherol | ● | | | ● | ● | |
| (Vitamin E) | | | | | | |
| Apotransferrin | | | | ● | | |
| Biotin | ● | | | ● | ● | |
| Beta mercaptoethanol | | | | | ● | |
| Calcium Pantothenate | ● | | | | | |
| Catalase | | | | ● | ● | |
| Corticosterone | | | | ● | ● | |
| Cyclodextrin | ● | | | | | |
| D-Galactose | | | | ● | ● | |
| DL-alpha-tocopherol | | | | ● | ● | |
| acetate | | | | | | |
| EGF | ● | | | | | |
| Ethanolamine | ● | | | ● | ● | |
| Exendin | ● | | | | | |
| Glutathione | ● | | | ● | ● | |
| HGF | ● | | | | | |
| IGF1 | ● | | | | | |
| Insulin | ● | | | ● | ● | ● |
| KGF | ● | | | | | |
| L Carnithine | | | | ● | ● | |
| Linoleic acid | | | | ● | ● | |
| Myoinositol | ● | | | | | |
| Nicotinamide | ● | | | | | |
| Progesterone | | | | ● | ● | ● |
| Prolactin | ● | | | | | |
| Putrescine | | | | ● | ● | ● |
| Retinyl acetate | | | | ● | ● | |
| Selenium | ● | | | ● | ● | ● |
| Super oxide | | | | ● | ● | |
| Dismutase | | | | | | |
| Transferrin | ● | | | ● | ● | ● |
| T3 | | | | ● | ● | |
| VEGF | ● | | | | | |
| ZnSO4 | ● | | | | | |
| Glucose | 10 mM | | | 12.6 mM | | |
| concentration | | | | | | |

*includes B27 sold by Gibco ™ as a serum free supplement for the culture of neurons.

Table 1 lists factors which may be added to the culture media which include potential growth factors and potential differentiation factors. Table 1 also lists components other than the basal media which may promote the cell culture. Table 2 lists supplements which may be added to the culture media. For purposes of this disclosure, the terms “factor”, “component” and “supplement” may be used interchangeably.

These components, factors and supplements include but are not limited to Tissue Culture water, ZnSO₄, HEPES buffer, Sodium Pyruvate, Insulin, transferrin, Ethanolamine, Human Serum Albumin, Sodium Selenite, Linoleic Acid, Oleic Acid, cyclodextrin, Biotin, Glutamine, α-Tocopherol, Calcium Pantothenate, Myoinositol, EGF, VEGF, FGF, PDGF, HGF Exendin, IGF-1, Glucose, Reg I, CCK, Pancreatic Polypeptide, Somatostatin, Prolactin, Placental Lactogen, Transforming Growth Factor β (TGF-β), B27, TGF, NGF, Nicotinamide, Secretin IGFII, Transforming Growth Factor α (TGF-α), KGF, PTHRP, Hepatocyte Growth Factor, Glucagon-like Peptide-1 (GLP-1), Fetal Bovine or Human Serum, Apotransferrin, Catalase, Corticosterone, D-galactose, DL-α-tocopherol acetate, DMSO, D-Raffinose, Glutathione, Glycine, Lactobionic acid, L-Carnitine, Magnesium sulfate, Nycodenz, Progesterone, Prolactin, Putrescine, Retinyl acetate, Selenium, Super oxide Dismutase, and T3. Preferably, the culture media contains at least one of the factors and supplements listed in Tables 1 and/or 2 and above. More preferably, the culture media contains at least two of the factors and supplements listed in Tables 1 and/or 2 and above. More preferably, the culture media contains at least three of the factors and supplements listed in Tables 1 and/or 2 and above. More preferably, the culture media contains at least four of the factors and supplements listed in Tables 1 and/or 2 and above. More preferably, the culture media contains at least five of the factors and supplements listed in Tables 1 and/or 2 and above. More preferably, the culture media contains at least six of the factors and supplements listed in Tables 1 and/or 2 and above. More preferably, the culture media contains at least seven of the factors and supplements listed in Tables 1 and/or 2 and above. More preferably, the culture media contains at least eight of the factors and supplements listed in Tables 1 and/or 2 and above. More preferably, the culture media contains at least nine of the factors and supplements listed in Tables 1 and/or 2 and above. More preferably, the culture media contains at least ten of the factors and supplements listed in Tables 1 and/or 2 and above. More preferably, the culture media contains at least eleven of the factors and supplements listed in Tables 1 and/or 2 and above. More preferably, the culture media contains at least twelve of the factors and supplements listed in Tables 1 and/or 2 and above. More preferably, the culture media contains at least thirteen of the factors and supplements listed in Tables 1 and/or 2 and above. More preferably, the culture media contains at least fourteen of the factors and supplements listed in Tables 1 and/or 2 and above. More preferably, the culture media contains at least fifteen of the factors and supplements listed in Tables 1 and/or 2 and above. More preferably, the culture media contains at least sixteen of the factors and supplements listed in Tables 1 and/or 2 and above. More preferably, the culture media contains at least seventeen of the factors and supplements listed in Tables 1 and/or 2 and above. More preferably, the culture media contains at least eighteen of the factors and supplements listed in Tables 1 and/or 2 and above. More preferably, the culture media contains at least nineteen of the factors and supplements listed in Tables 1 and/or 2 and above. More preferably, the culture media contains at least twenty of the factors and supplements listed in Tables 1 and/or 2 and above. More preferably, the culture media contains at least twenty one of the factors and supplements listed in Tables 1 and/or 2 and above. More preferably, the culture media contains at least twenty two of the factors and supplements listed in Tables 1 and/or 2 and above. The culture media may also contain more than twenty two of the factors and supplements listed in Tables 1 and/or 2 and above.

Any basal media may be used for the culturing described herein. Preferred basal media include Dulbecco's Modified Eagle's medium (DMEM), Medium 199 (M199), Ham's F12 Nutrient Mixture (Ham's F12), RPMI 1640 Medium, CMRL Medium, and combinations of the above. Preferably, the basal media is at least 15% by volume of the tissue culture media. Preferably, the basal media is at least 25% by volume of the tissue culture media. Yet more preferably, the basal media is at least 35% by volume of the tissue culture media. Yet more preferably, the basal media is at least 55% by volume of the tissue culture media. Yet more preferably, the basal media is at least 65% by volume of the tissue culture media. Yet more preferably, the basal media is at least 75% by volume of the tissue culture media. In a most preferred embodiment, the basal media is 45% by volume.

In some embodiments, more than one basal media is used. Combinations of 2, 3, or 4 different basal media are used in certain embodiments. Combinations of Dulbecco's Modified Eagle's medium (DMEM), Medium 199 (M199), Ham's F12 Nutrient Mixture (Ham's F12), RPMI 1640 Medium, and CMRL Medium are preferred. However, substitution of other known basal media for those listed is also encompassed herein.

In some embodiments, supplements are added to the basal media. These supplements are listed in Tables 1 and 2, and discussed above. The concentration ranges for these supplements may vary. Preferred concentration ranges are as follows: for Tissue Culture water a preferred concentration is at least 20% by volume; yet more preferred is 5% by volume; and yet more preferred is 10% by volume. For ZnSO₄, a more preferred concentration range is 0-5 μM; yet more preferred is 50-100 μM; and yet more preferred is 5-50 μM. For addition of HEPES buffer, a preferred concentration is 1-2 mM; yet more preferred is 20-50 mM; and yet most preferred is 2-20 mM. For addition of B27 (Gibco™), a preferred concentration is 0.1% - 1%; yet more preferred is 4%-50%; and yet most preferred is 1%-4%. For addition of Sodium Pyruvate, a preferred concentration is 0.1-0.5 mM; yet more preferred is 5-10 mM; most preferred is 0.5-5 mM. For insulin, a preferred concentration range is 0.01-1 mg/L; yet more preferred is 10-30 mg/L; yet most preferred is 1-10 mg/L. Other preferred concentration ranges for insulin are 2-5 mg/L and 5-8 mg/L. For human transferrin, a preferred concentration range is 0.1-0.5 mg/L; yet more preferred is 5-10 mg/L; yet most preferred is 0.5 to 5 mg/L. For ethanolamine a preferred concentration range is 0.05-0.1 mg/L; yet more preferred is 3-5 mg/L; yet most preferred is 0.1-3 mg/L. For human serum albumin a preferred concentration range is 2-10 mg/L, yet more preferred is 30-50 mg/L; yet most preferred is 10-30 mg/L. For sodium selenite a preferred concentration range is 0.05-0.2 μg/ml; yet more preferred is 5-10 μg/ml; yet most preferred is 0.2-5 μg/ml. For linoleic acid, a preferred concentration range is 0.05-0.2 μg/ml; yet more preferred is 5-10 μg/ml; yet most preferred is 0.2-5 μg/ml. For oleic acid, a preferred concentration range is 0.05-0.2 μg/ml; yet more preferred is 5-10 μg/ml; yet most preferred is 0.2-5 μg/ml. For cyclodextrin, a preferred concentration is 50-100 mg/ml; yet more preferred is 750-1500 mg/ml; yet most preferred is 100-750 mg/ml. For biotin, a preferred concentration is 0.01-0.05 μM; yet more preferred is 0.75-200 μM; yet more preferred is 0.05-0.75 μM. For glutamine a preferred concentration is 1-2 mM; yet more preferred is 15-40 mM; yet most preferred is 2-15 mM. For a-tocopherol, a preferred concentration is 1-2 IU/ml; yet more preferred is 50-100 IU/ml; yet most preferred is 2-50 IU/ml. For calcium pantothenate a preferred concentration is 1-5 mM; yet more preferred is 25-50 mM; yet most preferred is 5-25 mM. For myoinositol, a preferred concentration is 0.01-0.05 mM, yet more preferred is 0.75-1.5 mM; yet most preferred is 0.05 to 0.75 mM.

For Epidermal growth factor a preferred concentration is 0.05-0.1 ng/ml; yet more preferred is 50-100 ng/ml; yet most preferred is 0.1-50 ng/ml. Other preferred concentration ranges for epidermal growth factor include 0.1-10 ng/ml and 10-50 ng/ml. For Vascular Endothelial Growth Factor (VEGF) a preferred concentration range is 0.05-0.1 ng/ml; yet more preferred is 50-100 ng/ml; yet most preferred is 0.1-50 ng/ml. Other preferred concentration ranges for VEGF include 0.1-10 ng/ml and 10-50 ng/ml. For Fibroblastic Growth Factor (FGF) a preferred concentration is 0.05-0.1 ng/ml; yet more preferred is 50-100 ng/ml; yet most preferred is 0.1-50 ng/ml. Other preferred concentration ranges for FGF include 0.1-10 ng/ml and 10-50 ng/ml. For Platelet Derived Growth Factor (PDGF) a preferred concentration is 0.05-0.1 ng/ml; yet more preferred is 50-100 ng/ml; yet most preferred is 0.1-50 ng/ml. Other preferred concentration ranges for PDGF include 0.1-10 ng/ml and 10-50 ng/ml. For Hepatocyte Growth Factor (HGF) a preferred concentration is 0.05-0.1 ng/ml; yet more preferred is 50-100 ng/ml; yet most preferred is 0.1-50 ng/ml. Other preferred concentration ranges for HGF include 0.1-10 ng/ml and 10-50 ng/ml. For Insulin-like Growth Factor-1 (IGF-1) a preferred concentration is 0.05-0.1 ng/ml; yet more preferred is 50-100 ng/ml; yet most preferred is 0.1-50 ng/ml. Other preferred concentration ranges for IGF-1 include 0.1-10 ng/ml and 10-50 ng/ml.

For Exendin-4 a more preferred is 0.01-0.05 ng/ml; yet more preferred is 5-10 ng/ml; yet most preferred is 0.05-5 ng/ml. For glucose, a preferred concentration is 2-8 mM; yet more preferred is 20-50 mM; yet more preferred is 8-20 mM. For Reg I a preferred concentration is 0.05-0.1 ng/ml; yet more preferred is 50-100 ng/ml; yet most preferred is 0.1-50 ng/ml. Other preferred concentration ranges for Reg I include 0.1-10 ng/ml and 10-50 ng/ml. For Cholecystokinin (CCK) a preferred concentration is 0.05-0.1 ng/ml; yet more preferred is 50-100 ng/ml; yet most preferred is 0.1-50 ng/ml. Other preferred concentration ranges for CCK include 0.1-10 ng/ml and 10-50 ng/ml. For Pancreatic Polypeptide, a preferred concentration range is 0.01-1 mg/ml; yet more preferred is 20-50 mg/ml; yet most preferred is 1-20 mg/ml. For Somatostatin, a preferred concentration range is 0.01-1 mg/ml; yet more preferred is 20-50 mg/ml; yet most preferred is 1-20 mg/ml. For Prolactin, a preferred concentration range is 0.01-0.05 ng/ml; yet more preferred is 25-50 ng/ml; yet most preferred is 0.05-25 ng/ml. For Placental Lactogen, a preferred concentration range is 0.01-0.05 ng/ml; yet more preferred is 25-50 ng/ml; yet most preferred is 0.05-25 ng/ml.

For TGF-β a preferred concentration is 0.05-0.1 ng/ml; yet more preferred is 50-100 ng/ml; yet most preferred is 0.1-50 ng/ml. Other preferred concentration ranges for TGF-β include 0.1-10 ng/ml and 10-50 ng/ml. For Nerve Growth Factor (NGF) a preferred concentration is 0.05-0.1 ng/ml; yet more preferred is 50-100 ng/ml; yet most preferred is 0.1-50 ng/ml. Other preferred concentration ranges for NGF include 0.1-10 ng/ml and 10-50 ng/ml. For KGF a preferred concentration is 0.05-0.1 ng/ml; yet more preferred is 50-100 ng/ml; yet most preferred is 0.1-50 ng/ml. Other preferred concentration ranges for KGF include 0.1-10 ng/ml and 10-50 ng/ml.

For Nicotinamide, a preferred concentration range is 0.01-0.1 mM, yet more preferred is 5-10 mM; yet most preferred is 0.1-5 mM. For Secretin a preferred concentration is 0.05-0.1 ng/ml; yet more preferred is 50-100 ng/ml; yet most preferred is 0.1 -50 ng/ml. Other preferred concentration ranges for Secretin include 0.1-10 ng/ml and 10-50 ng/ml.

For Islet-like Growth Factor II (IGFII) a preferred concentration is 0.05-0.1 ng/ml; yet more preferred is 50-100 ng/ml; yet most preferred is 0.1-50 ng/ml. Other preferred concentration ranges for IGFII include 0.1-10 ng/ml and 10-50 ng/ml. For TGF-α a preferred concentration is 0.05-0.1 ng/ml; yet more preferred is 50-100 ng/ml; yet most preferred is 0.1-50 ng/ml. Other preferred concentration ranges for TGF-α include 0.1-10 ng/ml and 10-50 ng/ml. For Parathyroid Hormone Receptor Protein (PTHRP) a preferred concentration is 0.05-0.1 ng/ml; yet more preferred is 50-100 ng/ml; yet most preferred is 0.1-50 ng/ml. -Other preferred concentration ranges for PTHRP include 0.1-10 ng/ml and 10-50 ng/ml. For Hepatocyte Growth Factor a preferred concentration is 0.05-0.1 ng/ml; yet more preferred is 50-100 ng/ml; yet most preferred is 0.1-50 ng/ml. Other preferred concentration ranges for Hepatocyte Growth Factor include 0.1-10 ng/ml and 10-50 ng/ml. For Glucagon-like Peptide 1 (GLP-1) a preferred concentration is 0.05-0.1 ng/ml; yet more preferred is 50-100 ng/ml; yet most preferred is 0.1-50 ng/ml. Other preferred concentration ranges for Glucagon-like Peptide 1 (GLP-1) include 0.1-10 ng/ml and 10-50 ng/ml.

In a preferred embodiment, differentiated, non-hormone producing cells are cultured in adherent culture mode for 5-10 days in a first step in a first medium, followed by a second step of culture for an additional 7-21 days, preferably 7-14 days, in a second culture medium to obtain hormone-producing cells. Preferably, the culture media for the first step is selected from RPMI+10% FBS, Novocell Medium, RPMI 1640 Medium, RPMI 1640 Medium+10% FBS. Preferably, the culture medium for the second step is selected from Johe's N2 medium, Johe's N2 with additional FGF, Johe's N2 with additional nicotinamide, Novocell medium with and without additional insulin, Neurobasal medium plus supplements, Neurobasal medium plus supplements with additional nicotinamide, Neurobasal medium plus supplements plus additional FGF, and a mixture of DMEM/Ham's F12+10% FBS plus insulin, transferrin, selenium and fibronectin. In a most preferred embodiment, the culture medium is RPMI 1640 Medium+10% FBS for the first step with Novocell Medium for the second step.

In a preferred embodiment, differentiated, non-hormone producing cells are cultured in a first step in a suspension culture mode for 5-12 days in a first medium followed by culture in a second step for an additional 7-14 days, in a second culture medium which may be the same as the first culture medium, to obtain hormone-producing cells. Preferably, the culture media for the first step is selected from DMEM+10% FBS+geneticin and M199+2% HSA. Preferably, the culture medium for the first step is the same as the culture medium for the second step.

In a preferred embodiment, differentiated, non-hormone producing cells are cultured in a first step in suspension culture mode for 5-12 days in a first medium followed by a second step of embedding into a polymerizable gel, preferably Matrigel™ or alginate, and culturing for an additional 5-10 days, in a second culture medium, to obtain hormone-producing cells. In a preferred embodiment, the culture medium for the first step is RPMI+10% FBS. In a preferred embodiment, the culture medium for the second step is Novocell Medium.

In a preferred embodiment, differentiated, non-hormone producing cells are cultured in a first step in adherent culture mode for 10-18 days in a first medium followed by overlay with a polymerizable gel, preferably, Matrigel™ or alginate, and culture for an additional 5-10 days in a second step in a second culture medium, to obtain hormone-producing cells. Preferably, the culture media for the first step is selected from Novocell medium, RPMI+10% FBS, CMRL+10% FBS, and DMEM+10% FBS supplemented with insulin transferrin, selenium and genticin. Preferably, the culture medium for the second step is selected from Novocell medium, stem cell medium supplemented with additional KGF and HGF, stem cell medium supplemented with additional VEGF, DMEM+10% FBS supplemented with insulin, transferrin, selenium and geneticin. Preferably, the polymerizable gel is supplemented with laminin or hyaluronic acid. In an alternate embodiment, both step 1 and step 2 are carried out in a polymerizable gel.

Preferably, the determination that undifferentiated stem cells have been produced is performed by observation of the cell morphology. Preferably, the determination that undifferentiated stem cells have been produced is performed by the presence or absence of certain known cell markers. Preferably, cell replication is measured by an increase in DNA content.

Preferably, the determination that hormone-producing cells have been produced is indicated by observation of the cell morphology. Preferably, the determination that hormone-producing cells have been produced is indicated by the presence or absence of certain known cell markers. Preferably, the determination that hormone-producing cells have been produced is indicated by the ability of the cells to produce hormone. More preferably, the determination that insulin-producing cells have been produced is indicated by the ability of the cells to produce insulin. Preferably, insulin formation is assayed after a glucose challenge which corrects for any insulin that may be present in the culture media, and which is not the result of the formation of insulin-producing cells.

Preferred cell markers used in the practice of one aspect of the described invention include but are not limited to CK19, PCNA, Ki67, and PDX-1. In one embodiment, at least 10% of the stem cells express at least one of the following markers comprised of CK19, and PDX-1. Preferably, at least 20% of the stem cells express at least one of the following markers comprised of CK19, and PDX-1. More preferably, at least 30% of the stem cells express at least one of the following markers comprised of CK19, and PDX-1. In a most preferred embodiment, more than 30% of the stem cells express at least one of the following markers comprised of CK19, and PDX-1.

In another preferred embodiment, the invention provides methods and compositions for the large scale transformation of acinar cells into insulin producing islet cells. In another preferred aspect, the invention provides methods and compositions for the growth and expansion of acinar, duct and islet cells so as to maintain the cells in optimal health for dedifferentiation into stem cells, differentiation into hormone producing cells such as insulin, and transplantation. Another preferred embodiment of the invention is the identification and use of markers to characterize the phenotype of pancreatic cells at each step of expansion, dedifferentiation into stem cells and differentiation into insulin-producing cells.

In a preferred embodiment, insulin-producing cells produced by the methods described herein are implanted into a mammalian subject in need thereof. In one embodiment, they are implanted into a diabetic test animal. In one aspect, the diabetic test animal is an animal treated with streptozotocin to induce hyperglycemia. In another aspect, the test animal is an athymic diabetic test animal. In a preferred embodiment, the test animal is a mouse. In another preferred embodiment, the test animal is a diabetic primate.

In an alternate preferred embodiment, insulin-producing cells produced by the methods described herein are implanted into a human subject in need thereof, preferably a diabetic patient.

In another aspect, the present invention includes culture media as described herein.

In one embodiment, a culture media is described for the culture of islet cells. In a preferred embodiment, this culture media is Novocell Media.

Another preferred embodiment of the invention is a method of establishing and stabilizing pancreatic cells in suspension culture in preparation of expansion of the cells, comprising the steps of:

1. Directly culturing primary cells in suspension culture conditions in basic media supplemented from a list that includes but is not limited to FGF, EGF, VEGF, and PDGF, and insulin, selenium, steroids, glucose, glutamine, transferrin.

Another preferred embodiment of the invention is a method of establishing and stabilizing pancreatic cells in adherent culture in preparation of expansion of the cells, comprising the step of:

1. Directly culturing primary cells in adherent culture conditions from a list that includes but is not limited to collagen, laminin, fibronectin, alginate, in basic media supplemented with a list of factors that includes but is not limited to FGF, EGF, VEGF, and PDGF, and insulin, selenium, steroids, glucose, glutamine, transferrin.

Another preferred embodiment of the invention is a method of establishing and stabilizing pancreatic cells in adherent culture in preparation of expansion of the cells, comprising the steps of:

1. Placing the pancreatic cells in a matrix for three-dimensional support and anchorage dependency signals; and

2. Culturing the matrix-supported in basic media.

Another preferred embodiment of the invention is the large-scale expansion of acinar cells in suspension, adherent or matrix culture, comprising the step of:

1. culturing the acinar cells in NCBM media by supplementing the media with general growth factors from a list that includes but is not limited to FGF, EGF, PDGF, VEGF, and specific growth factors from a list that includes but is not limited to CCK, TGF-beta, and additives from a list that includes but is not limited to steroids, glucose, insulin, pancreatic polypeptide, somatostatin, glucagon.

Another preferred embodiment of the invention is the large scale conversion of acinar cells into stem cells that are differentiated into hormone-producing cells in suspension, adherent or matrix culture, comprising the steps of:

a) culturing the acinar cells in basic NCBM, RPMI 1640, Media 199 or Hams 12 media supplemented with factors as described in Tables 1 and 2, such as human serum replacement proteins.

Another preferred embodiment of the invention is the large scale conversion of differentiated non-hormone producing pancreatic cells e.g. acinar cells into stem cells followed by the further differentiation into hormone-producing cells in matrix culture, comprising the steps of:

a) culturing the acinar cells in basic NCBM, RPMI 1640, Media 199 or Hams 12 media supplemented with factors such as human serum replacement proteins.

b) adding general growth factors that includes but is not limited to FGF, EGF, PDGF, VEGF, NGF and specific growth factors that includes but is not limited to IGF1, IGF2, GLP1, nicotinamide, HGF, TGF-alpha, PTHRP, KGF, Secretin, and additional factors such as glucose, selenium, insulin, transferrin.

Another preferred embodiment of the invention is a method of converting the differentiated non-hormone producing pancreatic cells such as acinar cells into hormone-producing cells comprising the step of:

a) directly culturing primary cells in suspension culture conditions in basic media supplemented from a list that includes but is not limited to FGF, EGF, VEGF, and PDGF, and insulin, selenium, steroids, glucose, glutamine, transferrin.

Another preferred embodiment of the invention is a method of converting the acinar cells into insulin producing cells comprising the step of:

a) directly culturing primary cells in adherent culture conditions from a list that includes but is not limited to collagen, laminin, fibronectin, alginate, in basic media supplemented from a list that includes but is not limited to FGF, EGF, VEGF, and PDGF, and insulin, selenium, steroids, glucose, glutamine, transferrin.

Another preferred embodiment of the invention is a method of initiating the conversion of acinar cells into insulin producing cells comprising the step of:

a) placing cells in matrix for three-dimensional support and anchorage dependency signals and culture in basic media.

In another preferred embodiment the invention provides methods of expanding acinar cells in culture, such as expanding the acinar cells in NCBM media by supplementing the media with general growth factors from a list that includes but is not limited to FGF, EGF, PDGF, VEGF, and specific growth factors from a list that includes but is not limited to CCK, TGF-beta, and additives from a list that includes but is not limited to steroids, glucose, insulin, pancreatic polypeptide, somatostatin, glucagon. This expansion can take place in suspension, adherent or matrix based culture.

In another preferred embodiment, the invention provides a method of converting acinar cells into stem cells by culturing the cells in basic NCBM, RPMI 1640, Media 199 or Hams 12 media supplemented with factors such as human serum replacement proteins. This conversion can take place in suspension, adherent, or matrix based culture. If performed in matrix, additional differentiation along the duct cell lineage can be effected by the addition of general growth factors from a list that includes but is not limited to FGF, EGF, PDGF, VEGF, NGF and specific growth factors from a list that includes but is not limited to IGF1, IGF2, GLP1, nicotinamide, HGF, TGF-alpha, PTHRP, KGF, Secretin, and additional factors such as glucose, selenium, insulin, transferrin.

In another preferred embodiment, the invention provides methods of differentiating the stem cells from matrix culture to hormone-producing cells by adding factors, from a list that includes but is not limited to FGF, EGF, PDGF, VEGF and specific growth and differentiation factors from a list that includes but is not limited to IGF1, IGF2, nicotinamide, GLP1, exendin 4, HGF, TGF alpha to basic media.

In another preferred embodiment the invention provides a method of differentiating the stem cells from matrix culture to hormone-producing cells by culturing the cells under conditions that mimic those of differentiating neural cells from neural stem cells, such as using Johe's N2 media supplemented with growth factors from a list that includes but is not limited to FGF, EGF, NGF, PDGF, VEGF and specific growth factors from a list that includes but is not limited to nicotinamide, glucose, GLP1, exendin 4, Reg 1. The differentiation can be performed in suspension, adherence or matrix based culture.

In another preferred embodiment, the invention provides methods of moving hormone-producing cells expression onto complete insulin-producing beta cell expression by culturing the cells in basic media supplemented with specific growth and differentiation factors from a list that includes but is not limited to FGF, EGF, NGF, PDGF, VEGF, IGF1, IGF2, GLP1, exendin 4, prolactin, glucose, placental lactogen, growth hormone, HGF, TGF alpha, Reg 1. The final differentiation can take place in suspension, adherence, or matrix based culture.

In another preferred embodiment, the invention provides a method for attaching the cells to culture surfaces or matrices through the use of specific binding proteins or agents such as fibronectin, collagen, laminin, hyaluronic acid and other agents that permit an anchored cell state permitting more efficient cell expansion, and/or differentiation into insulin-producing cells.

In another embodiment, the invention provides compositions useful for the method of converting pancreatic acinar cells to stem cells that can differentiate into functional duct cells.

### EXAMPLES

### Example 1

### Composition of Culture Media

A variety of culture media are used throughout these studies each containing differing combinations of growth and differentiation factors. These media are summarized in Table 2.

### Example 2

### Conversion of Pancreatic Cells into Stem Cells in Adherent Culture

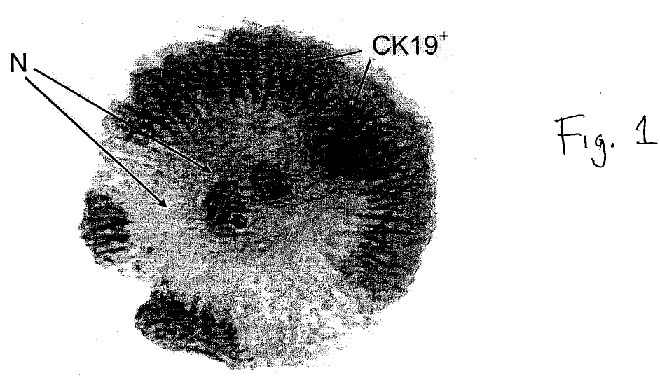

Pancreatic cells were cultured in adherent culture in DMEM+10% FBS supplemented with insulin, transferrin, selenium and geneticin. Non adherent cells were removed after 12 hours and medium changed and every 2-3 days thereafter. Cells were collected on day 0 and day 14, fixed in formalin and embedded in paraffin. CK19 expression was determined using monoclonal antibody staining and counterstained with hematoxylin. FIG. 1 demonstrates the presence of pancreatic cells with dual phenotype showing two nuclei (a characteristic of acinar cells) in conjunction with CK19 demonstrating the existence of cells with a combination of phenotypes.

Pancreatic cells were cultured in RPMI+10% serum in untreated polystyrene flasks. Culture density was 1.0 μl/cells per cm2 of tissue culture surface. Half the medium was replaced the following day, and again three days later. The population of cells that were adherent to the flask were collected and compared to cells that were collected on day 0. Samples were fixed in formalin, paraffin embedded, and sectioned. PCNA, CK19 and insulin expression was evaluated using monoclonal antibody staining; counterstaining was performed with hematoxylin.

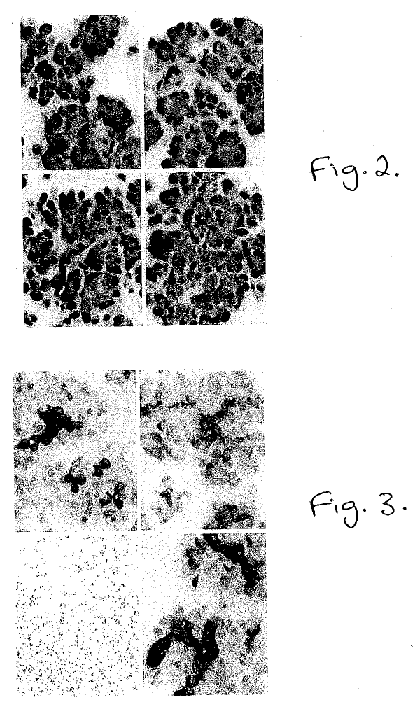

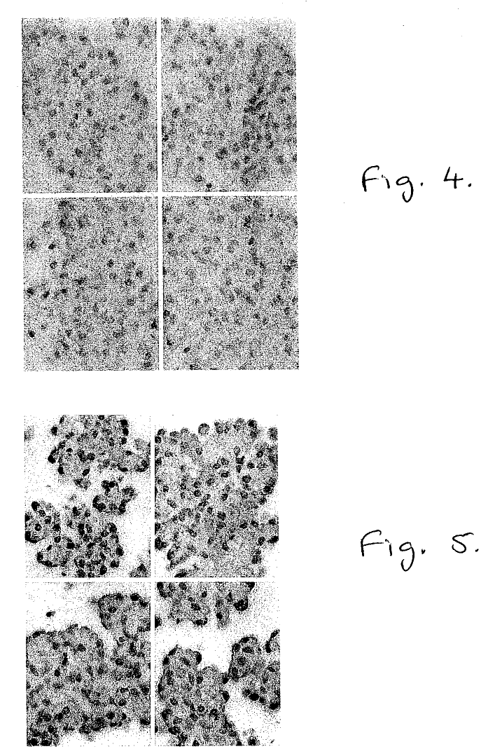

The phenotype of the day 0 material is shown in FIGS. 2-5. FIG. 2 illustrates the morphology using hematoxylin and eosin. FIG. 3 illustrates the morphology using CK19. FIG. 4 illustrates PCNA expression. FIG. 5 illustrates the insulin expression.

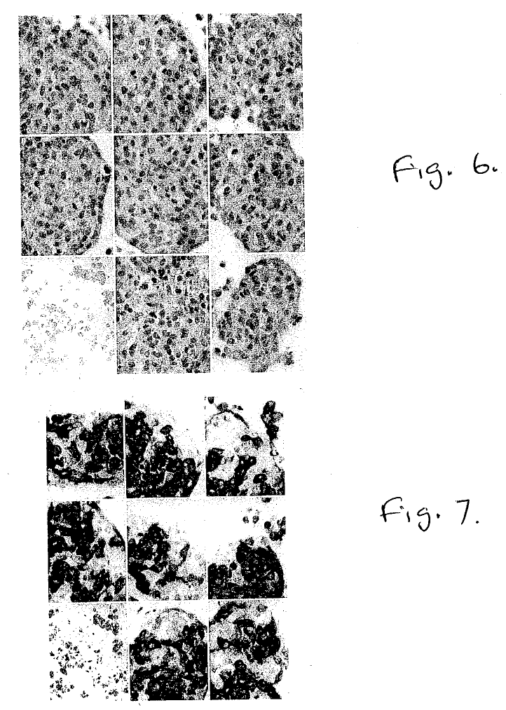

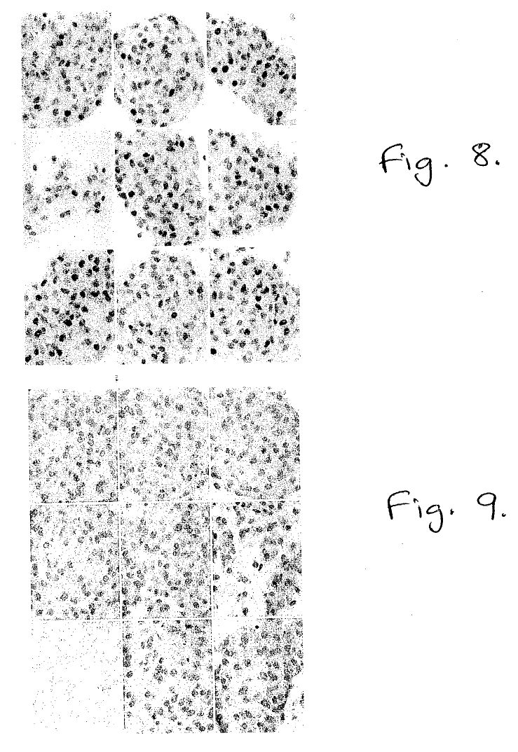

The phenotype of the material harvested on day 5 is shown in FIGS. 6-9. FIG. 6 illustrates the morphology using hematoxylin and eosin. FIG. 7 illustrates CK19 expression. FIG. 8 illustrates PCNA expression. FIG. 9 illustrates the insulin expression.

These data show a change in phenotype over the 5 day culture period as indicated by change in the hematoxylin and eosin pattern of staining; up regulation of CK19 and PCNA expression; and with no change in insulin expression.

### Example 3

### Proliferation of Pancreatic Cells Converted to Stem Cells in Adherent Culture

Pancreatic cells were cultured in different media for a period of up to 21 days in adherent culture and the percentage of proliferating cells determined at various time points using anti-Ki67 monoclonal antibody staining.

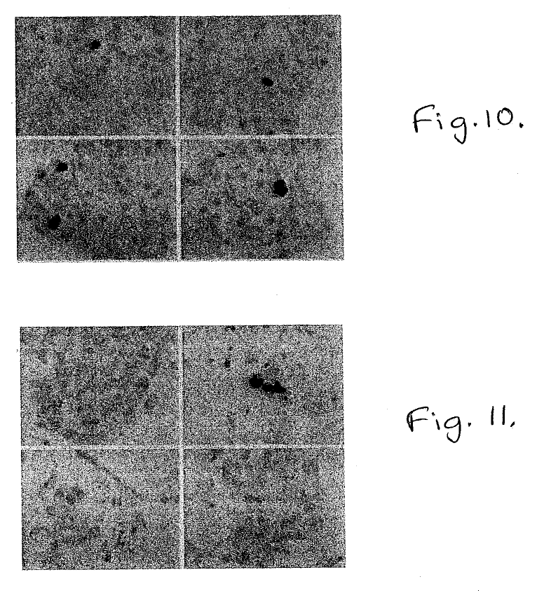

Data is shown in FIGS. 10-18 and results are summarized as follows.

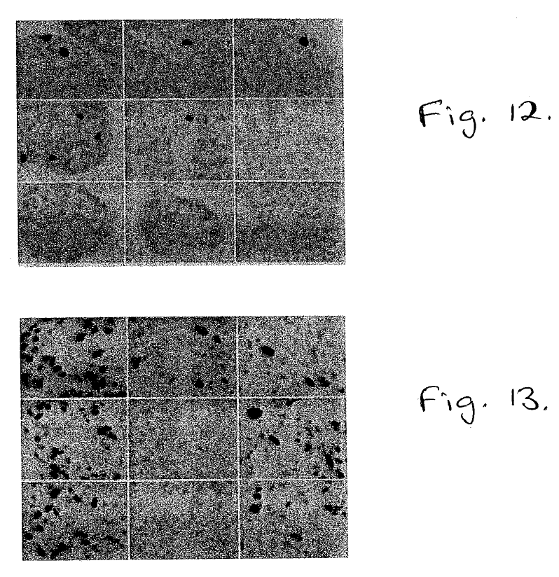

Day 0 material had a low level of Ki67 expression (FIG. 10) with no increase after culture for 7 days in RPMI+10% FBS (FIG. 11) or Novocell medium (FIG. 12).

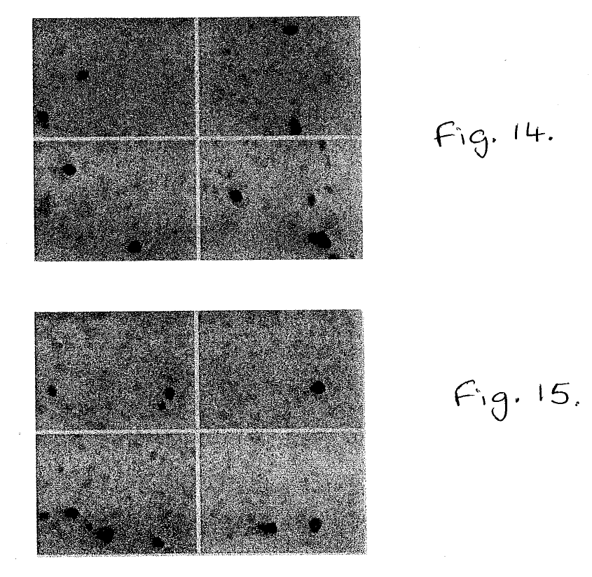

RPMT 1640 Medium+10% FBS for 7 days followed by Johe's N2 with additional FGF for 7 days: FIG. 13 illustrates that there was an increase in Ki67 staining at the 14 day time point compared to day 0.

Johe's N2 with additional FGF for 7 days followed by Johe's N2 with additional nicotinamide for 7 days: FIG. 14 illustrates that there was no change in Ki67 staining at the 14 day time point compared to day 0 with this treatment.

Novocell medium for 7 days, followed by Novocell medium with additional insulin for an additional 7 days: FIG. 15 illustrates that there was no change in the level of Ki67 staining compared to day 0.

RPMI 1640 medium+10% FBS for 7 days followed by Novocell Medium for 14 days: FIG. 16 illustrates that there was a significant increase in Ki67 staining under these conditions suggesting this formulation may a suitable medium for pancreatic and stem cell expansion.

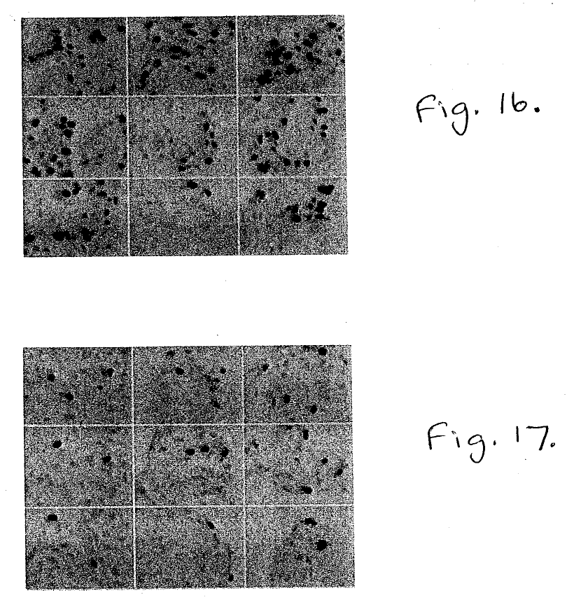

RPMI 1640 medium+10% FBS for 7 days, followed by Johe's N2 with additional FGF for 14 days: FIG. 17 illustrates that there was a slight increase in Ki67 positive staining compared to day 0.

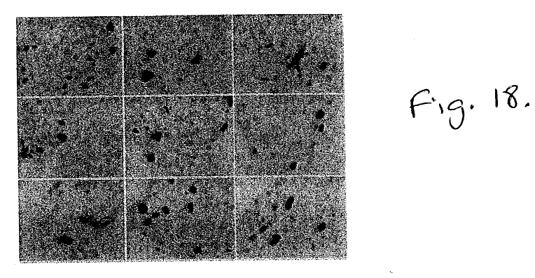

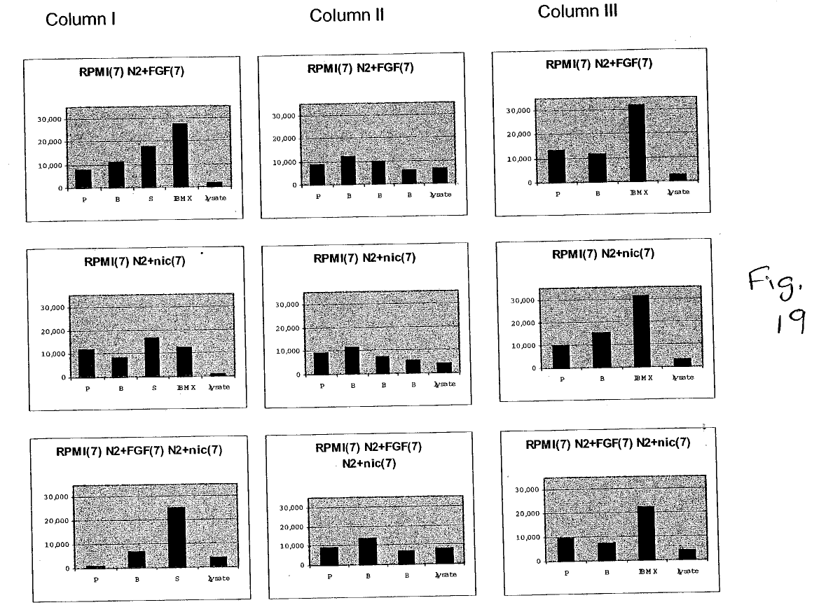

RPMI 1640 medium for 7 days, followed by Johe's N2 with additional FGF for 7 days, followed by Johe's N2 with additional nicotinamide for 7 days: FIG. 18 illustrates that there was a slight increase in Ki67 staining compared to day 0.

These data show that the proliferation of stem cells is variable and dependent upon the medium used to culture them. From these experiments sequential culture of pancreatic cells in RPMI 1640 medium+10% FBS for 7 days followed by and addition 14 day culture in Novocell medium was the optimal culture medium for the proliferation of these cells as determined by Ki67 staining.

### Example 4

### Primary Conversion of Pancreatic Cells into Stem Cells and then into Insulin Producing Cells in Adherent Culture

Pancreatic cells were cultured for 7 days in RPMI+10% FBS followed by an additional 7-14 day culture in Neurobasal medium plus supplements (Table 2). Cells were subjected to a routine static glucose challenge in the presence of basal medium (control) 20 mM glucose or 20 mM glucose in addition to 1 mM IBMX. Supernatants were collected and assayed for insulin and proinsulin content using a radioimmunoassay and cells were harvested (at each time point), lysed in 0.2% triton X-100 in TE buffer and DNA content determined using a pico green assay. Insulin:DNA and Proinsulin:DNA ratios were calculated (Table 3).

TABLE 3

| | Day 0 | Day 7 | Day 14 | Day 21 |
|---|---|---|---|---|
| Insulin/DNA | 0.0441 | 0.0107 | 0.0142 | 0.0398 |
| [ng/ng] | | | | |
| Proinsulin/DNA | 0.00192 | 0.00484 | 0.00519 | 0.02567 |
| [ng/ng] | | | | |

The insulin/DNA ratio dropped significantly by day 7 and 14 and returned to near starting levels by day 21. The proinsulin/DNA ratio increased by day 7 followed by a further increase by day 21 resulting in a 13.4 fold increase in proinsulin content over the 21 day culture period. This increase in proinsulin:DNA in the absence of a similar increase in insulin:DNA suggests that the stem cells have differentiated into an immature insulin producing cell that it not yet able to cleave the proinsulin molecule into insulin and c-peptide and transport it out of the cell. These data demonstrated that, using these culture conditions, the pancreatic cells convert into a stem cell population that can further differentiate into insulin producing precursors in culture.

### Example 5

### Primary Conversion of Pancreatic Cells into Stem Cells and then into Insulin Producing Cells in Adherent Culture

Pancreatic cells were cultured in RPMI 1640 medium+10% FBS for 7 days then changed to Johe's N2 medium with additional FGF or nicotinamide. After a further 7-14 days in culture, flasks were assayed for insulin release after a standard 24 hour glucose stimulation (SGS). Supernatants were collected and assayed for insulin content using a radioimmunoassay. Cells were harvested and lysed in 0.05% triton X-100 in TE and assayed for DNA content using a pico green assay or insulin content using a radioimmunoassay. FIG. 19 illustrates a summary of the data.

Column I: Cells were incubated in basal medium for 4 hour (P) when the medium was collected and replaced with basal medium for an additional 24 hours (B) and supernatants collected. This was followed by an additional 24 hour challenge with medium containing 12 mM glucose (S) when supernatants were collected followed by a final 24 hour challenge in the presence of 12 mM glucose in addition to 1 mM IBMX (I) and supernatants collected. Cells were then harvested, lysed and then assayed for DNA and insulin content.

Column II: Cells were subjected to the same sequence of challenges as in column I in the presence of basal medium alone i.e. one 4 hour challenge followed by 3 sequential 24 hour challenges. Cells were then harvested and lysed and the lysates assayed for DNA and insulin content.

Column III: Cells were incubated in basal medium for 4 hour (P) when the medium was collected and replaced with basal medium for an additional 24 hours (B) and supernatants collected. This was followed by a final 24 hour challenge in the presence of 12 mM glucose in addition to 1 mM IBMX (I) and supernatants collected. Cells were then harvested, lysed and then assayed for DNA and insulin content.

These data show that if cells are cultured for RPMI for 7 days followed by Johe's N2 medium+FGF for 7 days, the cells thus produced can be induced to produce insulin in response to glucose and/or IBMX. After an additional 7 day culture in Johe's N2 medium with additional nicotinamide, the cells are still responsive to these stimuli. In contrast cells cultured in RPMI for 7 days followed by Johe's N2 with additional nicotinamide are somewhat less responsive to a glucose challenge but can still release insulin in response to IBMX (column III).

Results comparing DNA content with total Insulin content (from lysed cells that had not been challenged with glucose or IBMX) are shown in Table 4.

TABLE 4
Insulin:DNA ratios of cultured pancreatic cells

| | | Insulin:DNA |
|---|---|---|
| | | ratio |
| Sample | Day | (ng/ng) |
| Day 0 | 0 | 0.065 |
| RPMI(7) | 7 | 0.042 |
| RPMI(7) Johe's N2 + FGF(7) | 14 | 0.046 |
| RPMI(7) Johe's N2 + nic (7) | 14 | 0.072 |
| RPMI(7) Johe's N2 + FGF(7) N2 + nic(7) | 21 | 1.15 |

Table 4 shows that the culture of pancreatic cells in sequential changes of RPMI, Johe's N2+FGF, and Johe's N2+nicotinamide medium resulted in a 17 fold increase in the insulin:DNA ratio from day 0 to day 21. The insulin:DNA (ng:ng) ratio of a normal islet is 1, so these data show that, using these culture conditions, the pancreatic cells convert into a stem cell population that can further differentiate into insulin producing cells in culture with a potency equivalent to a normal beta cell.

### Example 6

### Primary Conversion of Pancreatic Cells into Stem Cells and then into Insulin Producing Cells in Adherent Culture

First Step: Culture in RPMI+10% FBS (Days 0-7)

Pancreatic cells were cultured in RPMI+10% FBS for 7 days on untreated polystyrene flasks culture. Non adherent cells and culture medium were removed. Cells were harvested at particular time points (during the first and second steps) by scraping cells from the culture vessel surface using a cell scraper then lysed in 0.05% triton X-100 in TE buffer followed by sonication. DNA content determined using pico green. Insulin and proinsulin were measured by radioimmunoassay.

Both the insulin content and the proinsulin content dropped significantly from the starting material during this culture period. Although the amount of insulin and proinsulin was extremely variable in the starting material there was generally less insulin containing cells in the adherent cells than in the non-adherent cells after 7 days in culture and significantly less than in the day 0 material.

Second Step: Culture in Neurobasal or Johe's N2 Medium (Days 8-21)

Adherent cells were placed into Johe's N2 medium or Neurobasal medium+supplements, in the presence or absence of additional growth factors, for two additional 7 day culture periods, as shown below, after which they were harvested for analyses.

a) N2+EGF (Days 8-14); N2 Alone (Days 15-21)

Cells from step one were cultured in N2+FGF (days 8-14) followed by N2 alone (days 15-21) for an additional 7 days (days 8-14).

Table 5 shows the insulin:DNA and proinsulin:DNA ratios.

TABLE 5

| | Day 0 | Day 7 | Day 14 | Day 21 |
|---|---|---|---|---|
| Insulin:DNA | 0.46 | 0.51 | 0.42 | 1.27 |
| (μIU/pg) | | | | |
| Proinsulin:DNA | 0.05 | 0.160 | 0.24 | 0.172 |
| (pmole:pg) | | | | |

Insulin:DNA ratios increased 2.5 times and the Proinsulin:DNA ratios increased 3.4 times over the 21 day culture period.

b) N2+Nic (Days 8-21)

Cells from step one were cultured in Johe's N2 medium with additional nicotinamide (days 8-21).

Table 6 shows the insulin:DNA and proinsulin:DNA ratios.

TABLE 6

| | Day 0 | Day 7 | Day 14 | Day 21 |
|---|---|---|---|---|
| Insulin:DNA | 0.46 | 0.51 | 0.31 | 1.09 |
| (μIU/pg) | | | | |
| Proinsulin:DNA | 0.05 | 0.160 | 0.06 | 0.18 |
| (pmole:pg) | | | | |

Insulin:DNA increased 2.1 times and the Proinsulin:DNA ratios increased 3.6 times over the 21 day culture period.

c) Neurobasal+Nic (Days 8-21)

Cells from step one were cultured in Neurobasal medium plus supplements with additional nicotinamide (days 8-21).

Table 7 shows the insulin:DNA and proinsulin:DNA ratios.

TABLE 7

| | Day 0 | Day 7 | Day 14 | Day 21 |
|---|---|---|---|---|
| Insulin:DNA | 0.46 | 0.51 | 0.04 | 0.04 |
| (μIU/pg) | | | | |
| Proinsulin:DNA | 0.05 | 0.160 | 0.008 | 0.019 |
| (pmole:pg) | | | | |

The insulin:DNA decreased 12.8 times and proinsulin:DNA ratios decreased 2.6 times over the 21 day culture period.

d) Neurobasal+FGF (Days 8-21)

Cells from step one were cultured in Neurobasal medium plus supplements with additional FGF.

Table 8 shows the insulin:DNA and proinsulin:DNA ratios.

TABLE 8

| | Day 0 | Day 7 | Day 14 | Day 21 |
|---|---|---|---|---|
| Insulin:DNA | 0.94 | 0.14 | 0.11 | 0.98 |
| (μIU/pg) | | | | |
| Proinsulin:DNA | 0.018 | 0.008 | 0.05 | 0.05 |
| (pmole:pg) | | | | |

There was no overall net change in insulin:DNA ratio content over the 21 day culture period but the proinsulin:DNA ratio increased 2.8 times over the same time frame

e) DMEM/Ham's F12+10% FBS+ITS+Fibronectin (Days 8-14), N2+FGF (days 15-21) N2 Alone (Days 22-28)

Cells from step one were cultured in a mixture of DMEM and Ham's F12 nutrient mixture+10% FBS supplemented with additional insulin, transferrin, selenium and fibronectin for days 8-14, Johe's N2 medium supplemented with additional FGF for days 15-21, and Johe's N2 alone for days 22-28.

Table 9 shows the insulin:DNA ratios.

TABLE 9

| | Day 0 | Day 7 | Day 14 | Day 21 | Day 28 |
|---|---|---|---|---|---|
| Insulin:DNA | 0.03 | 0.02 | 0.05 | 0 | 0.61 |
| (μIU/pg) | | | | | |

There was an overall twenty fold increase in insulin:DNA ratio content over the 28 day culture period

Experiments were undertaken on several occasions and statistical analysis undertaken using a Friedman test. These data demonstrated a significant increase in insulin:DNA ratios after the culture period on a consistent basis (α=0.046).

### Example 7

### Conversion and Expansion of Pancreatic Cells into Stem Cells in Suspension Culture

Pancreatic cells were placed into untreated polystyrene flasks (so that cells did not attach but remained in suspension) in DMEM+10% FBS+gencticin. Samples were collected on day 0 and after 5 days in culture, fixed in formalin and embedded in paraffin. Samples were stained for CK19, PCNA, insulin, glucagons and amylase using monoclonal antibody staining.

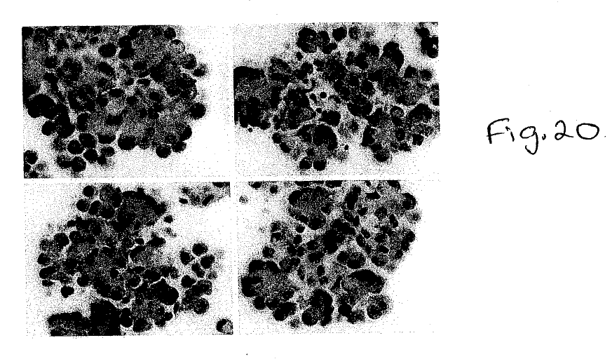

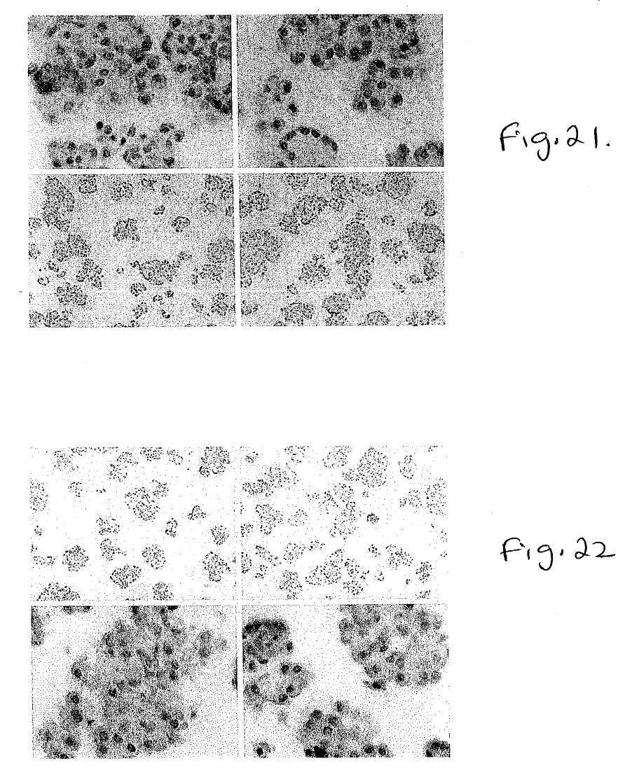

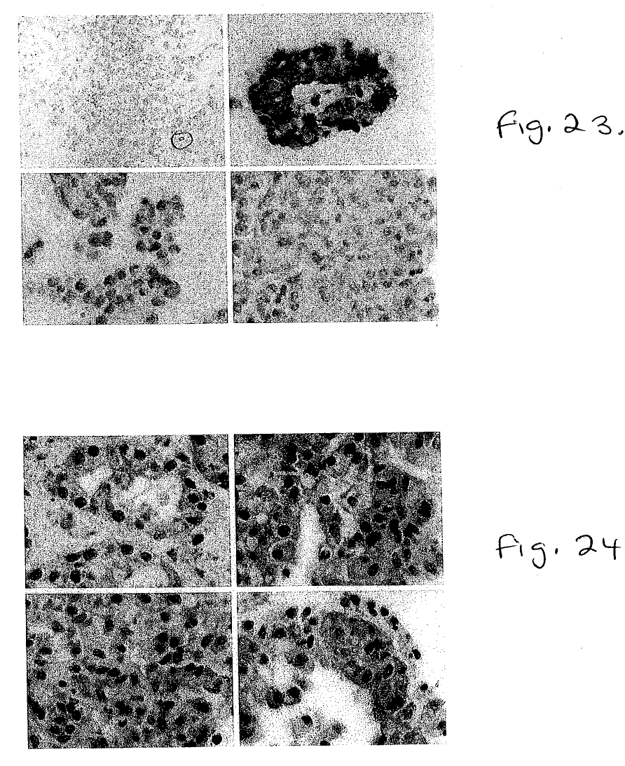

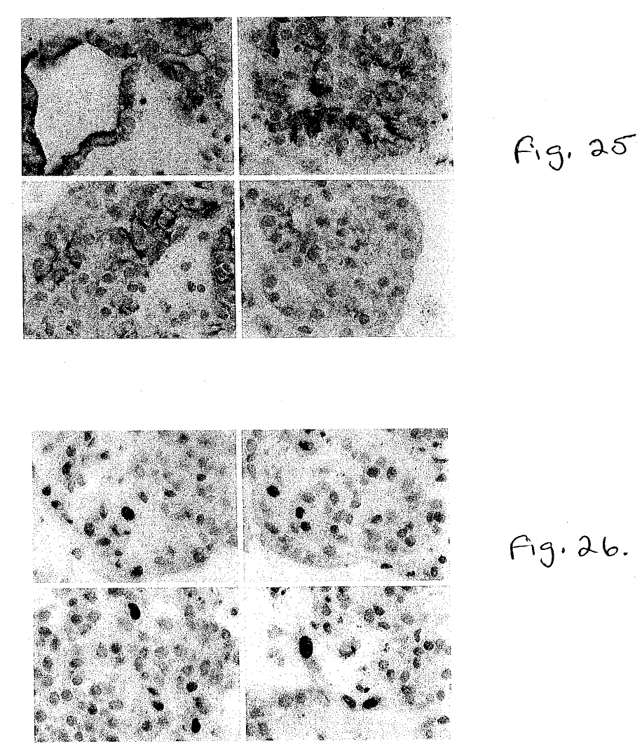

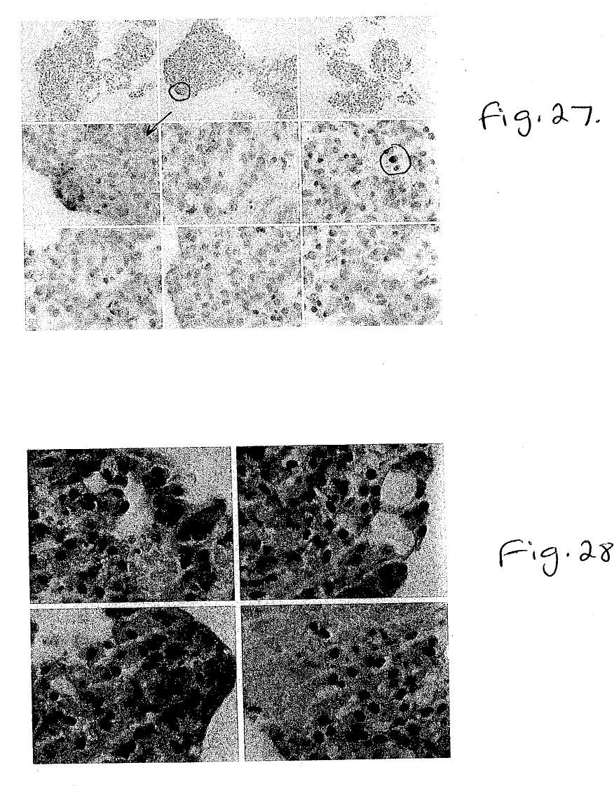

On day 0 cells had an aggregate morphology (FIG. 20), were amylase positive, CK19 (FIG. 21), and PCNA negative (FIG. 22), with few insulin (FIG. 23) and glucagon positive cells. On day 5 the cells exhibited significant changes in morphology (FIG. 24), were CK19 positive (FIG. 25) with many PCNA positive cells (FIG. 26), a few glucagon positive cells and rare amylase and insulin positive cells (FIG. 27).

These data indicate that the pancreatic cells transdifferentiated into a stem cell population with novel phenotypes accompanied by an expansion of these cells as indicated by an increase in the numbers of PCNA positive cells.

### Example 8

### Conversion of Pancreatic Cells into Insulin Producing Cells in Suspension Culture

Pancreatic cells were placed into bacteriological flasks (so that cells did not attach but remained in suspension) in M199+2% HSA. Samples were collected on day 0 and after 17 days in culture, fixed in formalin and embedded in paraffin. Samples were stained for CK19, PCNA, insulin, glucagon and amylase using monoclonal antibody staining.

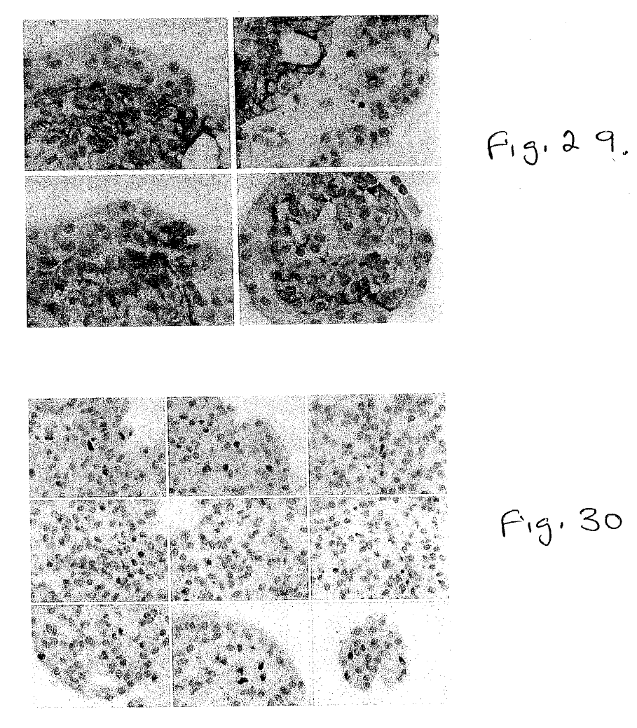

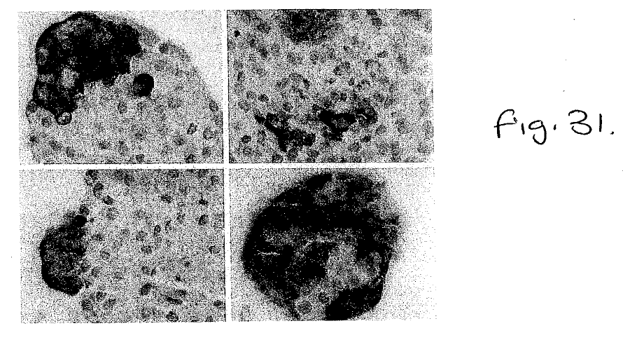

On day 0 cells had an aggregate morphology (FIG. 20), were amylase positive, CK19 (FIG. 21) and PCNA negative (FIG. 22), with few insulin (FIG. 23) and glucagon positive cells. On day 17 cells exhibited distinct changes in morphology (FIG. 28) many cells were CK19 (FIG. 29), and PCNA positive (FIG. 30), and a few cells were insulin positive (FIG. 31). Amylase and glucagon staining was not determined at this time point.

### Example 9

### Primary Conversion of Pancreatic Cells into Stem Cells and then into Duct Cells in Suspension and Matrix Culture

Pancreatic cells were cultured in suspension for 7 days in RPMI+10% FBS then cultured for an additional 7 day period embedded into polymerized MATRIGEL in Novocell medium. Day 0, day 7 and day 14 samples were collected and fixed in formalin then embedded in paraffin. Sections were stained for amylase, CK19, PCNA, pdx-1 and insulin using monoclonal antibody staining.

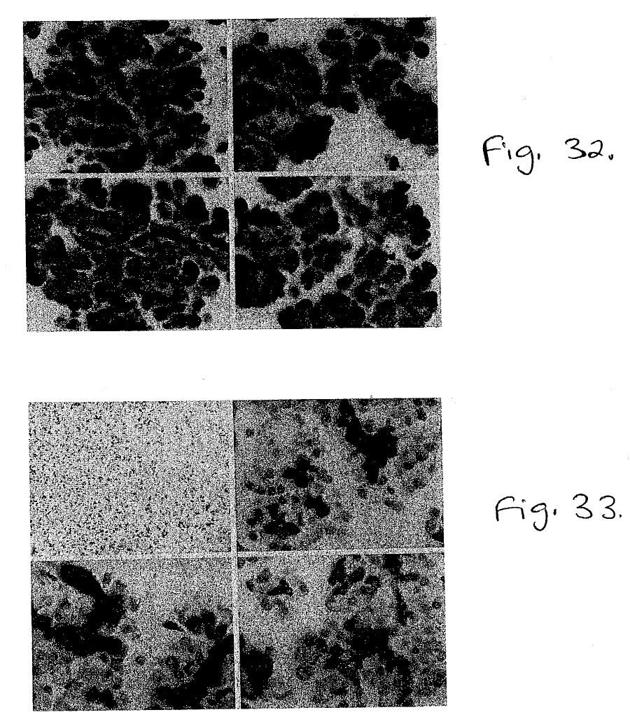

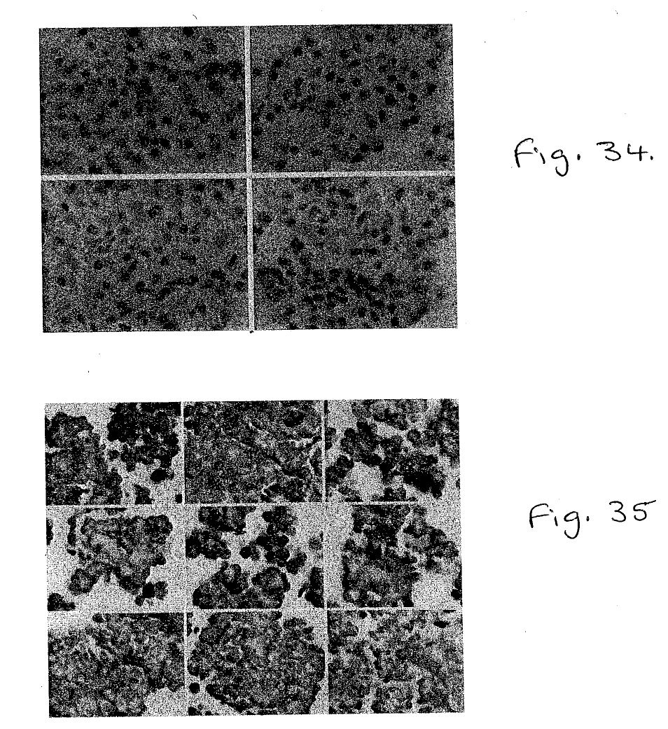

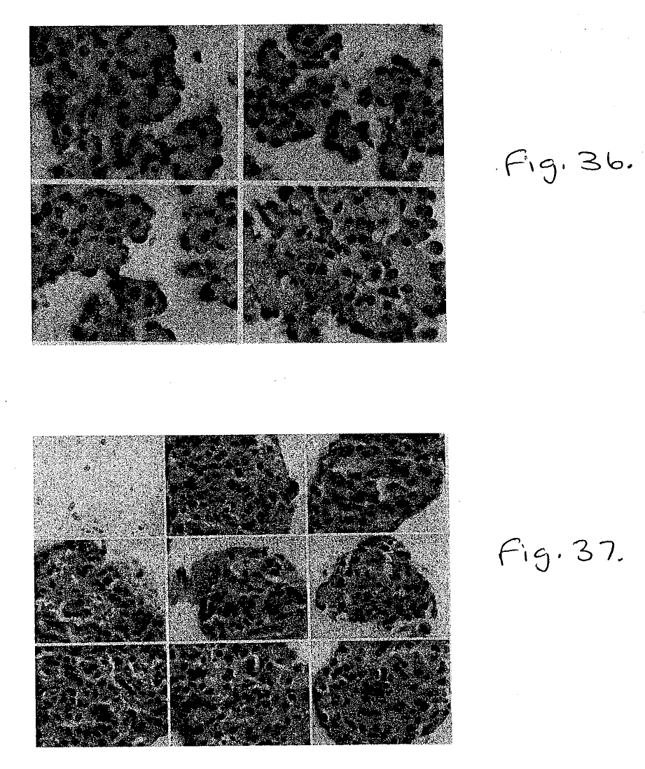

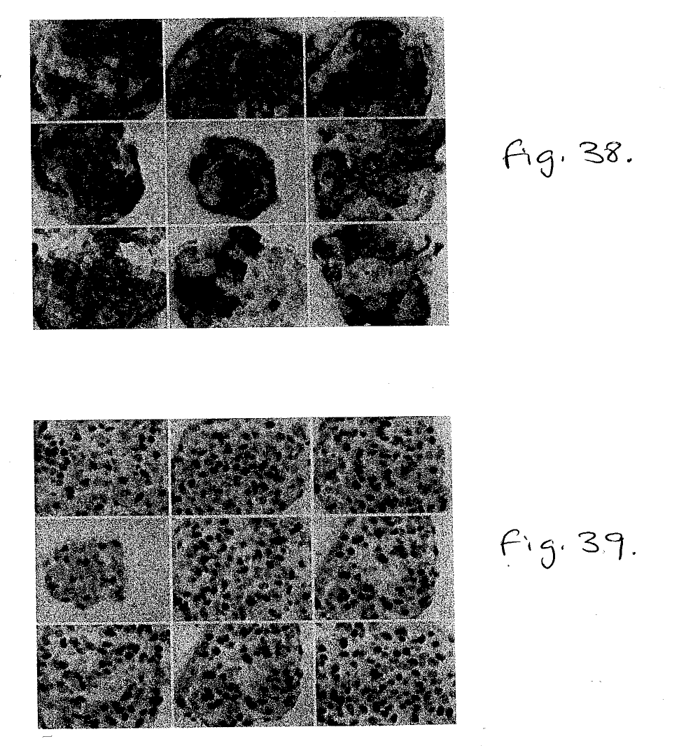

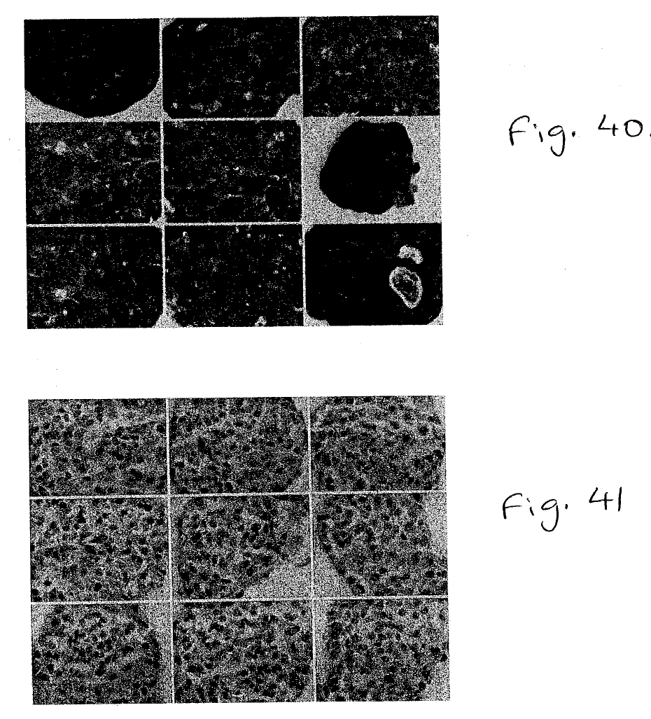

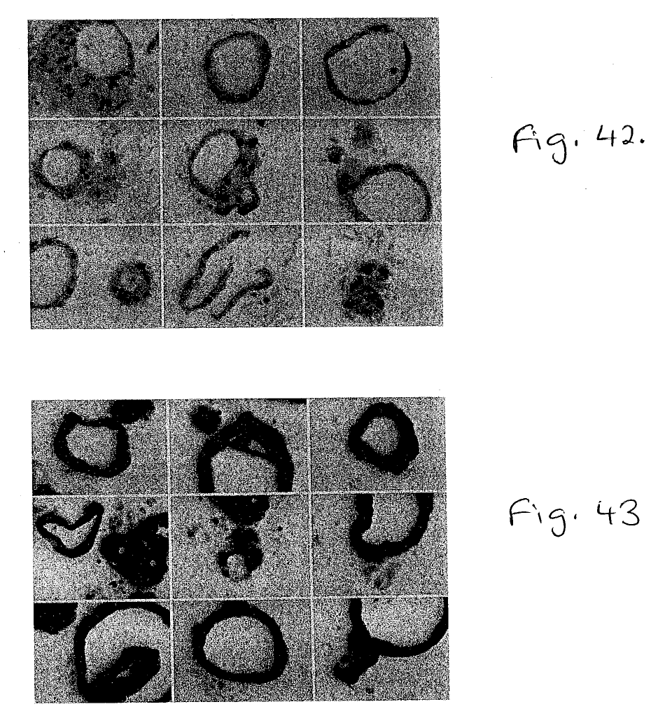

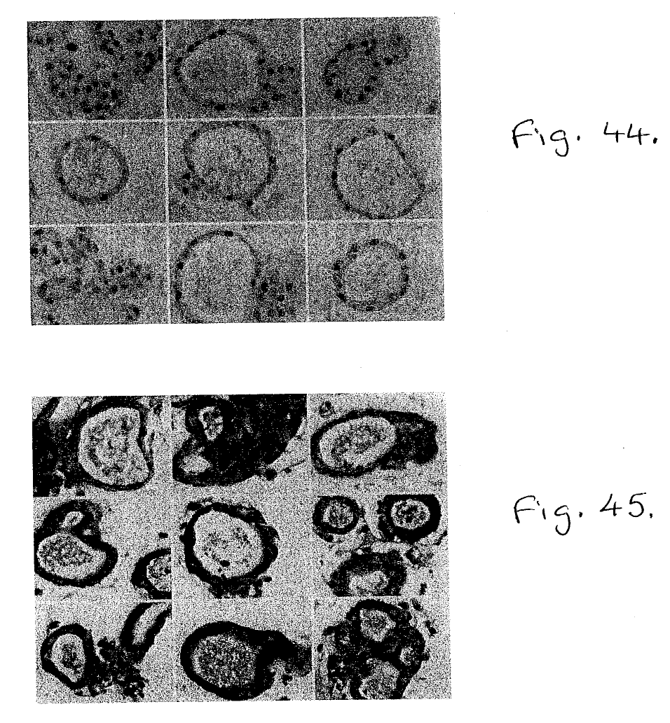

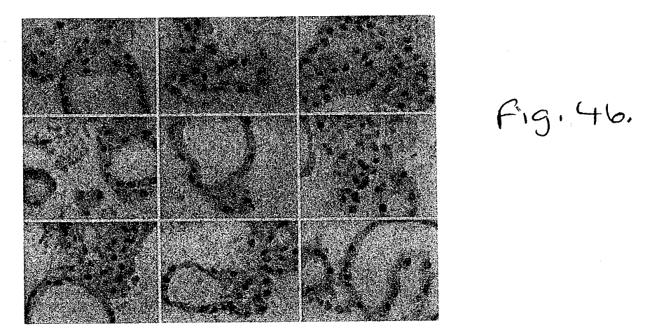

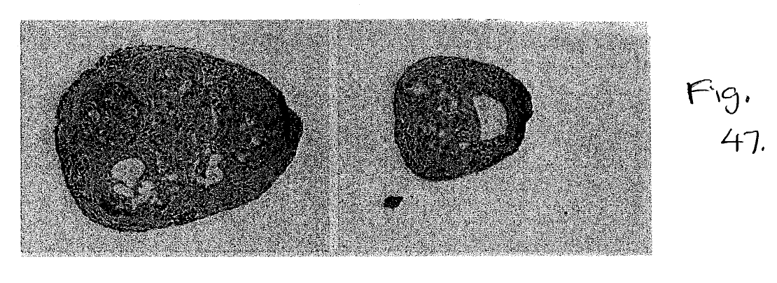

On day 0 the material had an aggregate morphology (FIG. 32) stained positive for amylase, a low percentage of cells were positive for CK19 (only in small ducts) (FIG. 33), a low level of PCNA (FIG. 34) with variable pdx-1 staining (FIG. 35) and negative for insulin (FIG. 36). After 7 days of culture, the cell aggregates remaining in suspension demonstrated changes in morphology (FIG. 37), significantly converted to CK19 (FIG. 38); PCNA (FIG. 39) and pdx-1 (FIG. 40) positive cells while the insulin content decreased (FIG. 41), if it was present initially. On day 14 the aggregate material formed cystic duct structures (FIG. 42) that contained CK19 positive cells (FIG. 43), which retained some degree of PCNA positive cells (FIG. 44), and a high percentage of pdx-1 positive cells (FIG. 45), and no insulin positive cells (FIG. 46). The changes were also seen if the starting cells were placed directly into MATRIGEL in Novocell Medium.

These data showed that pancreatic cells can convert to stem cells that can, in turn, differentiate into ductal structures.

### Example 10

### Method for the Production of Alginate Beads for the Culture of Pancreatic Cells

Islet depleted pancreatic cells were mixed with alginate (at a concentration of between 0.8 and 1.6% (v/v)) over a concentration range of between 5×10⁴ and 5×10⁷ cells/mL alginate. Two alternative methods of culturing the cells in alginate used: either in a “slab” of alginate or in alginate beads. When alginate was used as a “slab” liquid alginate/cell mixture was placed onto the surface of a tissue culture flask and polymerized by the addition of 80 mM CaCl₂ for a period of up to 60 minutes to form a “slab” of alginate in which the cells are suspended. When the alginate was used as beads the liquid alginate/cell mixture was dripped or sprayed into a beaker of 80 mM CaCl₂ resulting in the formation of alginate beads in which cells were trapped/suspended. The polymerized alginate (slab or beads) was washed twice in HBSS and overlaid or placed in culture medium as appropriate.

In order to harvest cells from the polymerized alginate, the alginate slab or beads were washed twice in HBSS for 5 minutes and the alginate depolymerized by the addition of citrate at a volume of between 5-20 times the volume of the slab/beads (over a concentration range between 27.5 and 55 mM) for a period of up to 60 minutes with agitation. The depolymerized alginate was diluted 1 to 2 in HBSS and mixed and the cells recovered from the depolymerized suspension by centrifugation and washed twice in HBSS.

### Example 11

### Recovery of Pancreatic Cells from Alginate Beads

Pancreatic cells were diluted in different concentrations of alginate and polymerized in beads by dropping into 80 mM CaCl₂. Beads were immediately depolymerized in 36 mM, cells washed in HBSS and lysed in 0.2% triton X-100. DNA content was determined using a pico green assay. Results from at least six replicates are as follows. DNA concentration (ng/bead): 0.8%=77.05±71.4; 1.0%=44.65±11.95; 1.2%=93.07±28.65

These data show that there is considerable variation in the distribution of cells between beads which is due to the aggregate nature of the material but there is no significant difference in the recovery of cells from beads made from different concentrations of alginate.

### Example 12

### Feasibility of Performing a Static Glucose Challenge on Insulin Producing Cells Embedded in a Polymerized Alginate Matrix

Cells from impure fractions of an islet isolation that are known to contain approximately 20% islets were embedded into polymerized gel of a 1.2% alginate and cultured in CMRL medium+10% FBS for 24 hours in a 96 well plate. Alginate beads were washed and the cell subjected to a 24 hour glucose challenge in the presence of basal medium control (basal) 20 mM glucose (glucose) or 20 mM glucose plus 1 mM IBMX (IBMX). Supernatants were harvested and assayed for insulin and c-peptide using an ELISA. Alginate beads were depolymerized, cells were washed and then lysed in 0.2% triton-X100 in TE buffer. Cell lysates were assayed for DNA content using pico green.

Results from a representative experiment, which contained 20% islets, showed a induced release of insulin by pancreatic cells [Insulin:DNA—ng/well:ng/well]—0.0367±0.0145 (B), 0.0753±0.0163 (G), and 0.1073±0.0364 (I); and an induced release of c-peptide by pancreatic cells [C-peptide:DNA—ng/well:ng/well]—0.0367±0.0168 (B), 0.0887±0.0186 (G), and 0.0986±0.0393 (I).

These data demonstrated that the alginate matrix system can be utilized in studies of insulin and c-peptide release using a standard glucose challenge. Studies are facilitated by the use of a 96 well plate for these assays.

### Example 13

### Feasibility of Depolymerizing of Alginate Beads in 96 Well Plates

Alginate (1.6%) beads were prepared in three different sizes by dropping liquid alginate into the polymerization solution (CaCl₂) using a 22 G or 18 G needle, or a 5 ml pipette. Beads thus produced had a volume of 12.5, 18.5 or 35 μl respectively. Individual beads were dispensed into wells of a 96 well plate with 300 μl citrate and the time taken to depolymerize noted. Beads dissolved in 35, 45 and 60 minutes respectively.

These data show that individual beads can be depolymerized in a 96 well plate, within a reasonable time frame, provided that the well volume does not exceed more than 10% of the volume of citrate used to perform the depolymerization process. Studies are facilitated by the use of a 96 well plate for a variety of assays.

### Example 14

### Expansion of Pancreatic Cells and Stem Cells in Alginate Culture

Acinar cells were cultured in a polymerized alginate (1.2% w/v) gel formed into beads at a cell density of 1:120 in three different culture media: Novocell medium, RPMI+10% FBS and CMRL+10% FBS. Beads were harvested on day 0, 1 7 and 14, depolymerized, cells harvested and lysed and DNA content determined using a pico green assay. Results are shown in Table 10.

TABLE 10
Influence of culture medium on the growth of pancreatic cells in polymerized alginate matrix

| | DNA concentration (ng DNA/bead) | | | | | | | | | |
|---|---|---|---|---|---|---|---|---|---|---|
| | Day 0 | | Day 1 | | Day 4 | | Day 7 | | Day 14 | |
| Medium | Mean | sd | Mean | sd | Mean | sd | Mean | sd | Mean | sd |
| Novocell | 84.26 | 26.82 | 258.16 | 56.95 | 140.40 | 16.44 | 103.28 | 17.64 | 43.70 | 10.46 |
| medium | | | | | | | | | | |
| RPMI + 10% | 84.26 | 26.82 | 252.01 | 78.14 | 111.29 | 14.93 | 99.98 | 29.07 | 48.31 | 21.67 |
| FBS | | | | | | | | | | |
| CMRL + 10% | 84.26 | 26.82 | 250.95 | 12.54 | 126.89 | 24.04 | 79.90 | 25.66 | 35.38 | 8.98 |
| FBS | | | | | | | | | | |

This representative experiment shows that there is no significant difference in the viability of pancreatic cells when cultured in different media.

Replicate experiments demonstrated that although the magnitude of cell expansion varied from preparation to preparation, the degree of expansion was not significantly affected by any particular media type.

Data also demonstrated that the level of expansion reached a plateau from days 1-7 (depending on the preparation) where the cells did not continue to replicate in the alginate matrix. This would be an optimal time in order to change the media environment to medium more suitable for differentiation as opposed to cell expansion

### Example 15

### Culture of Pancreatic Cells for Insulin Production in Alginate Matrix

Pancreatic cells were cultured in adherent culture in DMEM+10% FBS supplemented with insulin, transferring, selenium and geneticin for 14 days when they were evaluated for growth and phenotype. Cultures showed an extensive outgrowth of large flat cells with prominent nuclei. Adherent monolayers were then overlaid with polymerizable gels of collagen, MATRIGEL or alginate supplemented with laminin 5, hyaluronic acid or laminin 1 and cultured in Novocell medium, stem cell medium supplemented with additional KGF and HGF or VEGF alone, or DMEM+10% FBS supplemented with insulin, transferrin, selenium and geneticin as above. Control cells were cultured in these different media types in the absence of an overlay. After an additional 7 days in culture the cells were evaluated visually for growth and phenotype. Results are summarized in Table 11.

TABLE 11

| | | | | Alginate supplemented with: | | |
|---|---|---|---|---|---|---|
| | No | | | | Hyaluronic | |
| Medium | overlay | Collagen | matrigel | Laminin 5 | acid | Laminin 1 |
| DMEM + 10% FBS + ITS | +++ | − | ++ | + | − | + |
| Stem cell medium + KGF | ++ | − | + | + | + | +/− |
| and HGF | | | | | | |
| Stem cell medium + VEGF | + | − | + | − | − | +/− |
| Novocell | ++ | − | + | + | ++ | + |

Key: +++ excellent viability; ++ good viability; + poor viability; ± variable viability; − no cells viable

These data demonstrate that excellent viability can be maintained for up to 21 days, in the absence of overlay, in DMEM+10% FBS supplemented with insulin, transferrin and selenium. When overlays of polymerizable gels were used MATRIGEL was the most superior followed by alginate supplemented with laminin 5. Collagen overlay is deleterious to cell viability and stem cell medium supplemented with VEGF is not a suitable medium for the culture of these cells.

### Example 16

### Conversion of Stem Cells into Duct Cells by Implantation into a Diabetic Athymic Mouse

Pancreatic cells were cultured for 7 days in RPMI+10% FBS+geneticin and the cells harvested and implanted under the kidney capsule and into the peritoneal space of athymic diabetic mice. Kidneys and peritoneal washes were explanted at 30, 60 and 90 days, fixed in formalin and stained with hematoxylin and eosin or anti-CK19 monoclonal antibody.

FIG. 47 illustrates ductal structures, presumed to be of implant origin, were identified in the intraperitoneal washes explanted after 30 days. Implanted material was not identified under the kidney capsule of any mouse.

### Example 17

### Establishment of an Immunocompromised Diabetic Mouse Model

In previous studies conducted with normal and athymic mice, it has been established that a single high dose injection of Streptozotocin of 180 mg/kg i.v. induces hyperglycemia (>400 mg/dL) in mice within 7-10 days. These animals are then monitored for a period of at least 21 days prior to transplantation to ensure a true diabetic state. Once confirmed to be diabetic (blood glucose level >500 mg/dL and unresponsive to a GTT) the blood glucose levels are then kept at approximately normal levels using a surgically implanted Linbits.

Two separate groups of scid and rag-1 mice were purchased and, upon arrival, were quarantined for a period of 3 days to allow time for acclimatization. The mice were then injected with 180 mg/kg of Streptozotocin (STZ). Mice were considered completely diabetic if fasting blood glucose levels were >500 mg/dL and did not respond in a GTT; they were considered to be mildly diabetic if fasting blood glucose levels were approximately 300 mg/dL and did respond in a GTT. A total of 35 SCID mice were treated with STZ of which 32 mice died and 8 became completely diabetic. Two mice became mildly diabetic and 3 remained non-diabetic. A total of 25 Rag-1 mice were treated with STZ with a 100% survival rate. 17 mice became totally diabetic, two mice were mildly diabetic and six remained non-diabetic.

The rag-1 mouse model is a more suitable recipient of implanted material than the scid mouse since it has a higher success rate of inducing diabetes and has a higher post surgery survival rate.

### Example 18

### Detection of Human/Primate c-Peptide in Mouse Urine

Experiments were conducted to investigate if it is possible to detect human/primate c-peptide in murine urine.. Pooled murine urine derived from normal mice and mice implanted with baboon islets were diluted and spiked with different concentrations of human c-peptide standard. Samples were assayed for c-peptide content using a c-peptide ELISA

The level of human/primate c-peptide in normal mouse urine was found to be <0.15 ng/mL. When normal mouse urine was spiked with 7.5 ng/ml human c-peptide and subsequently assayed for c-peptide a concentration of 9.485±2.11 ng/mL was determined. When the urine from mice implanted with baboon islets was assayed for c-peptide and assayed for c-peptide a concentration of 0.55 ng/mL was detected. When this same urine was spiked with human c-peptide at 7.25 and 0.25 ng/ml and subsequently assayed for c-peptide a concentration of6.27±0.62 and—0.47±0.23 ng/mL respectively was detected.

These data show that human/primate c-peptide does not occur naturally in murine urine but human c-peptide can be detected in urine when it is added artificially. Factors present in the urine interfere with c-peptide detection. Primate c-peptide is present in the urine of mice implanted baboon with islets so it is, therefore, feasible to detect human/primate c-peptide that is added artificially or occurs naturally in an experimental animal.

The absolute concentration of c-peptide cannot be determined accurately for two reasons: Firstly there is some interference of factors present in the mouse that either decrease or elevate measured levels depending on the urine dilution; secondly, the amount of c-peptide present in any one urine sample at any time will be influenced by the physiology of the individual animal. Nevertheless, this method can be utilized to determine if human or primate cells, implanted into an immunocompromised mouse are producing c-peptide (and by inference, insulin) without sacrificing the animal or subjecting them to a GTT (oral glucose tolerance test).

### Example 19

### Culture of Islet Cells in Novocell Medium

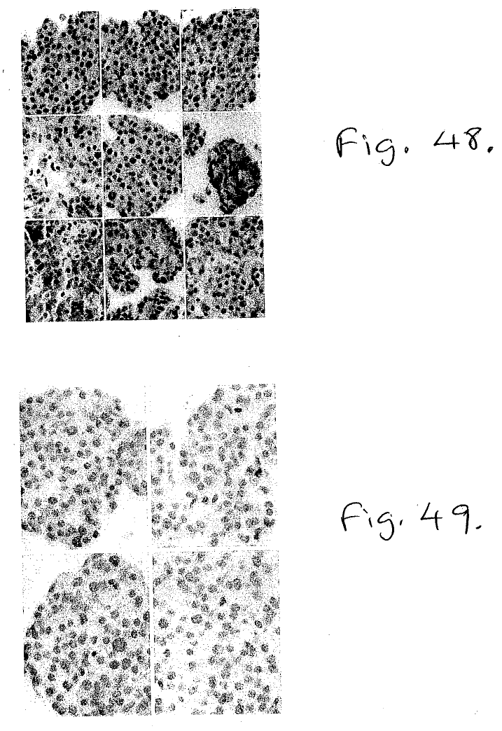

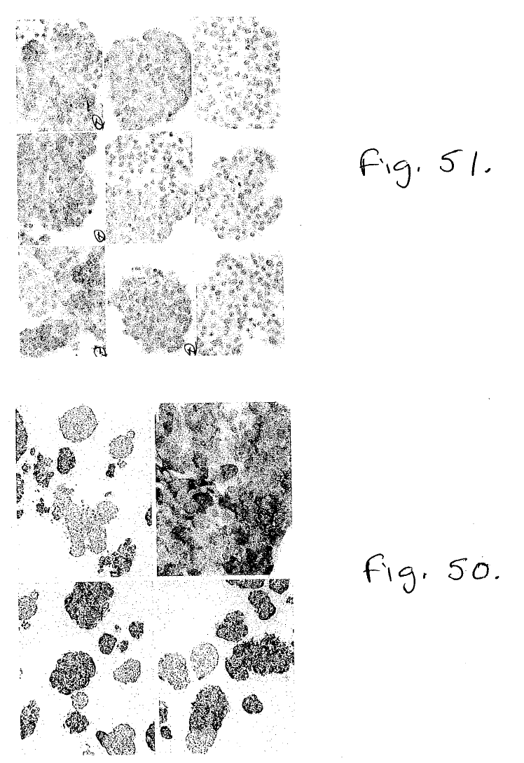

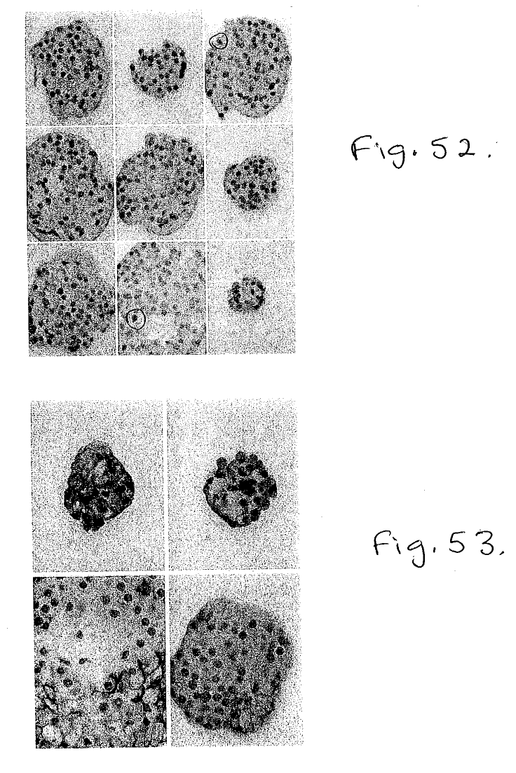

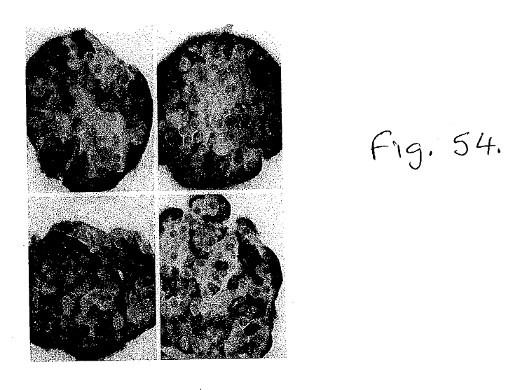

Upon receipt, human islet aggregates were cultured in Novocell medium, in standard tissue culture flasks. Cell density was 133 islets per cm². (Approximately 10,000 islets were placed into a T75 flask, with an islet comprising approximately 1,000 cells). Samples were taken for histology on day zero and after twenty five days in culture. Samples were fixed in formalin, embedded in paraffin, and sectioned. PCNA, CK19 and insulin expression was evaluated using monoclonal antibody, staining; counterstaining was performed with hematoxylin. On day zero cells exhibited islet morphology (FIG. 48), PCNA staining was negative (FIG. 49), some cells were positive for insulin (FIG. 50) and the majority of the cells were negative for CK19 (FIG. 51). On day zero, any CK19 positive cells were weakly positive. On day 25, a few rare cells were PCNA positive (FIG. 52), many cells were CK19 positive (FIG. 53) and many of the aggregates stained intensely for insulin (FIG. 54). These results suggest that Novocell medium maintains the beta cell phenotype of differentiated beta cells and supports the transdifferentiation of stem cells (carrying the CK19 phenotype) to the beta cell phenotype.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A method of converting differentiated non-hormone producing pancreatic cells into differentiated hormone-producing cells, comprising:
a) culturing said differentiated non-hormone producing pancreatic cells in a first cell culture system with a first cell culture medium, under conditions which provide for converting said differentiated non-hormone producing pancreatic cells into stem cells; and
b) culturing said stem cells in a second cell culture system with a second cell culture medium under conditions which provide for differentiating said stem cells into hormone-producing cells.

2. The method of claim 1, wherein said stem cells proliferate in said first step.

3. The method of claim 1, wherein said stem cells proliferate in said second step.

4. The method of claim 1, wherein said hormone-producing cells produce insulin.

5. The method of claim 1, wherein said hormone-producing cells produce glucagon.

6. The method of claim 1, wherein said differentiated non-hormone producing pancreatic cells are acinar cells.

7. The method of claim 1,
a) wherein said differentiated non-hormone producing pancreatic cells in the first step are cultured with a culture mode selected from the group consisting of: adherent, suspension and matrix; and
b) wherein said stem cells in said second step are cultured with a culture mode selected from the group consisting of: adherent, suspension, and matrix.

8. The method of claim 7, wherein said culture mode is an adherent culture mode that comprises culturing cells directly on a surface of a tissue culture container or on a surface of a tissue culture container which is coated with at least one compound selected from the group consisting of collagen, fibronectin, laminin, and hyaluronic acid.

9. The method of claim 1, wherein said differentiated non-hormone producing pancreatic cells are seeded at a density of 10⁵ to 10⁷ cells/cm².

10. The method of claim 7, wherein said culture mode is a suspension culture mode that comprises culturing said differentiated non-hormone producing pancreatic cells in suspension in said culture medium.

11. The method of claim 7, wherein said culture media is a matrix culture mode that comprises culturing said differentiated non-hormone producing pancreatic cells interspersed in a crosslinked polymerizable gel.

12. The method of claim 1, wherein said differentiated non-hormone producing pancreatic cells are seeded at a density of 10⁴ to 10⁸ cells/ml in a hydrogel.

13. The method of claim 12, wherein said hydrogel is alginate.

14. The method of claim 1, wherein said culture medium in the first step comprises serum and a basal medium selected from the group consisting of Dulbecco's Modified Eagle's medium (DMEM), Medium 199 (M199), Ham's F12 Nutrient Mixture (Ham's F12), RPMI 1640 Medium, CMRL medium, and mixtures thereof.

15. The method of claim 14, wherein said culture medium in the first step further comprises at least three compounds selected from the group consisting of insulin, transferrin, selenium, zinc sulphate, glutathione, ethanolamine, cyclodextrin, biotin, alpha Tocopherol, calcium pantothenate, myoinositol, nicotinamide, IGF1, Prolactin, exendin-4, EGF, VEGF, KGF, and HGF.

16. The method of claim 1, wherein said culture medium in the first step comprises a basal medium without serum selected from the group consisting of Dulbecco's Modified Eagle's medium (DMEM), Medium 199 (M 199), Ham's F12 Nutrient Mixture (Ham's F12), RPMI 1640 Medium, CMRL medium, and mixtures thereof.

17. The method of claim 16, wherein said culture medium in the first step further comprises at least three compounds selected from the group consisting of insulin, transferrin, selenium, zinc sulphate, glutathione, ethanolamine, cyclodextrin, biotin, alpha Tocopherol, calcium pantothenate, myoinositol, nicotinamide, IGF1, Prolactin, exendin, EGF, VEGF, KGF, and HGF.

18. The method of claim 1, wherein said culture medium in the second step comprises a basal medium without serum selected from the group consisting of Dulbecco's Modified Eagle's medium (DMEM), Medium 199 (M199), Ham's F12 Nutrient Mixture (Ham's F1 2), RPMI 1640 Medium, CMRL medium, neurobasal medium, Johe's N2 medium, and mixtures thereof.

19. The method of claim 18, wherein said culture medium in the second step further comprises insulin, transferrin, and selenium.

20. The method of claim 14, wherein said culture medium in the second step further comprises at least two compounds selected from the group consisting of glutathione, ethanolamine, biotin, alpha Tocopherol (Vitamin E), and albumin (human or bovine).

21. The method of claim 19, wherein said culture medium in the second step further comprises at least two compounds selected from the group consisting of glutathione, ethanolamine, biotin, alpha Tocopherol (Vitamin E), and albumin (human or bovine).

22. The method of claim 18, wherein said culture medium in the second step further comprises at least two compounds selected from the group consisting of L-carnitine, corticosterone, D(+) galactose, linoleic acid, linolenic acid, progesterone, putrescine, retinly acetate, triodo-1-thyronin (T3), DL-α-tocopherol acetate, catalase, superoxide dismutase, apotransferrin and bFGF.

23. The method of claim 19, wherein said culture medium in the second step further comprises at least two compounds selected from the group consisting of L-carnitine, corticosterone, D(+) galactose, linoleic acid, linolenic acid, progesterone, putrescine, retinly acetate, triodo-1-thyronin (T3), DL-α-tocopherol acetate, catalase, superoxide dismutase, apotransferrin and bFGF.

24. The method of claim 21, wherein said culture medium in the second step further comprises at least two compounds selected from the group consisting of L-carnitine, corticosterone, D(+) galactose, linoleic acid, linolenic acid, progesterone, putrescine, retinly acetate, triodo-1-thyronin (T3), DL-α-tocopherol acetate, catalase, superoxide dismutase, apotransferrin and bFGF.

25. A method of converting differentiated non-hormone producing pancreatic cells into stem cells comprising culturing said differentiated non-hormone producing pancreatic cells in a cell culture system with a cell culture medium, under conditions which provide for converting said differentiated non-hormone producing pancreatic cell into stem cells.

26. The method of claim 25, wherein said stem cells proliferate.

27. The method of claim 25, wherein the differentiated non-hormone producing pancreatic cells comprise pancreatic acinar cells.

28. The method of claim 27, wherein the acinar cells are in a pancreatic cell mixture.

29. The method of claim 25, wherein said differentiated non-hormone producing pancreatic cells are cultured with a culture mode selected from the group consisting of: adherent, suspension and matrix.

30. The method of claim 29, wherein said culture mode is an adherent culture mode that comprises culturing the pancreatic cell mixture directly on a surface of a tissue culture container or on a surface of a tissue culture container which is coated with at least one compound selected from the group consisting of collagen, fibronectin, laminin, and hyaluronic acid.

31. The method of claim 25, wherein said cells are seeded at a density of 10⁵ to 10⁷ cells/cm².

32. The method of claim 29, wherein said culture mode is a suspension culture mode that comprises culturing said pancreatic cell mixture in suspension in said culture medium.

33. The method of claim 29, wherein said culture mode is a matrix culture mode that comprises culturing said pancreatic cell mixture interspersed in a crosslinked polymerizable gel.

34. The method of claim 33, wherein said pancreatic cell mixture is seeded at a density of 10⁴ to 10⁸ cells/ml in a hydrogel.

35. The method of claim 34, wherein said hydrogel is alginate.

36. The method of claim 25, wherein said culture medium comprises serum and a basal medium selected from the group consisting of Dulbecco's Modified Eagle's medium (DMEM), Medium 199 (M199), Ham's F12 Nutrient Mixture (Ham's F12), RPMI 1640 Medium, CMRL medium, and mixtures thereof.

37. The method of claim 36, wherein said culture medium in the first step further comprises at least three compounds selected from the group consisting of insulin, transferrin, selenium, zinc sulphate, glutathione, ethanolamine, cyclodextrin, biotin, alpha Tocopherol, calcium pantothenate, myoinositol, nicotinamide, IGF1, Prolactin, exendin, EGF, VEGF, KGF, and HGF.

38. The method of claim 25, wherein said culture medium in the first step comprises a basal medium without serum selected from the group consisting of Dulbecco's Modified Eagle's medium (DMEM), Medium 199 (M199), Ham's F12 Nutrient Mixture (Ham's F12), RPMI 1640 Medium, CMRL medium, and mixtures thereof.

39. The method of claim 38, wherein said culture medium in the first step further comprises at least three compounds selected from the group consisting of insulin, transferrin, selenium, zinc sulphate, glutathione, ethanolamine, cyclodextrin, biotin, alpha Tocopherol, calcium pantothenate, myoinositol, nicotinamide, IGF1, Prolactin, exendin, EGF, VEGF, KGF, and HGF.

40. A method of converting stem cells into differentiated hormone-producing cells, comprising culturing the stem cells in a cell culture system with a cell culture medium whereby said stem cells are differentiated into hormone-producing cells wherein said culture medium comprises basal medium without serum and at least three compounds selected from the group consisting of glutathione, ethanolamine, biotin, alpha Tocopherol (Vitamin E), albumin (human or bovine), L-carnitine, corticosterone, D(+) galactose, linoleic acid, linolenic acid, progesterone, putrescine, retinly acetate, triodo-1-thyronin (T3), superoxide dismutase, apotransfeitin and bFGF.

41. The method of claim 40, wherein said stem cells proliferate.

42. The method of claim 40, wherein said hormone-producing cells produce insulin.

43. The method of claim 40, wherein said hormone-producing cells produce glucagon.

44. The method of claim 40, wherein said stem cell is cultured with a culture mode selected from the group consisting of: adherent, suspension and matrix.

45. The method of claim 44, wherein said culture mode is an adherent culture mode that comprises culturing stem cells directly on a surface of a tissue culture container or a surface of a tissue culture container which is coated with at least one compound selected from the group consisting of collagen, fibronectin, laminin, and hyaluronic acid.

46. The method of claim 40, wherein said stem cells are seeded at a density of 10⁵ to 10⁷ cells/cm².

47. The method of claim 44, wherein said culture mode is a suspension culture mode that comprises culturing said stem cells in suspension in said culture medium.

48. The method of claim 44, wherein said culture mode is a matrix culture mode that comprises culturing the stem cells interspersed in a crosslinked polymerizable gel.

49. The method of claim 48, wherein said stem cells are seeded at a density of 10⁴ to 10⁸ cells/ml in a hydrogel.

50. The method of claim 49, wherein said hydrogel is alginate.

51. The method of claim 40, wherein said basal medium without serum is selected from the group consisting of Dulbecco's Modified Eagle's medium (DMEM), Medium 199 (MI 99), Ham's F12 Nutrient Mixture (Ham's F12), RPMI 1640 Medium, CMRL medium, neurobasal medium, Johe's N2 medium, and mixtures thereof.

52. The method of claim 5 1, wherein said culture medium further comprises insulin, transferrin, and selenium.

53. The method of claim 51, wherein said culture medium comprises at least two compounds selected from the group consisting of glutathione, ethanolamine, biotin, alpha Tocopherol (Vitamin E), and albumin (human or bovine).

54. The method of claim 52, wherein said culture medium comprises at least two compounds selected from the group consisting of glutathione, ethanolamine, biotin, alpha Tocopherol (Vitamin E), and albumin (human or bovine).

55. The method of claim 51, wherein said culture medium further comprises at least two compounds selected from the group consisting of L-carnitine, corticosterone, D(+) galactose, linoleic acid, linolenic acid, progesterone, putrescine, retinly acetate, triodo-1-thyronin (T3), α-tocopherol, catalase, superoxide dismutase, apotransferrin and bFGF.

56. The method of claim 52, wherein said culture medium further comprises at least two compounds selected from the group consisting of L-carnitine, corticosterone, D(+) galactose, linoleic acid, linolenic acid, progesterone, putrescine, retinly acetate, triodo-1-thyronin (T3), α-tocopherol, catalase, superoxide dismutase, apotransferrin and bFGF.

57. The method of claim 54, wherein said culture medium further comprises at least two compounds selected from the group consisting of L-carnitine, corticosterone, D(+) galactose, linoleic acid, linolenic acid, progesterone, putrescine, retinly acetate, triodo-1-thyronin (T3), α-tocopherol, catalase, superoxide dismutase, apotransferrin and bFGF.
